(19) United States
(12) Patent Application Publication
(10) Pub. No.: US 2004/0247624 A1
(43) Pub. Date: Dec. 9, 2004

(54) Methods of making pharmaceutical formulations for the delivery of drugs having low aqueous solubility

(76) Inventors: Evan Charles Unger, Tucson, AZ (US); VaradaRajan Ramaswami, Tucson, AZ (US); Reena Zutshi, Tucson, AZ (US); Rachel Yvonne LaBell, Vail, AZ (US); Elizabeth Anne Pigman, Tucson, AZ (US)

Correspondence Address: REED & EBERLE LLP, 800 MENLO AVENUE, SUITE 210, MENLO PARK, CA, 94025, US

(21) Appl. No.: 10/456,193

(22) Filed: Jun. 5, 2003

(51) Int. Cl. A61K 9/00

(52) U.S. Cl. 424/400

(57) ABSTRACT

Methods are provided that for producing aqueous formulations of pharmaceutical agents having low aqueous solubility. The methods also provide a simple means of producing the formulation as a sterile product. The drug is physically entrapped by a spatially stabilized matrix comprising a hydrophilic or hydrophilic-hydrophobic block polymer, without being covalently bound to the polymer. The drug formulation is a nanoparticle or sub-nanoparticle in size. In a preferred embodiment the nanoparticles are anisotropic, being much longer than they are wide.

### TECHNICAL FIELD

The present invention relates to methods of producing aqueous formulations of pharmaceutical agents having low aqueous solubility. In particular, the invention relates to methods of producing such formulations by physically entrapping a drug by a spatially stabilized matrix, for example, comprising a hydrophilic or hydrophilic-hydrophobic block polymer, where the drug is not covalently bound to the stabilizing material. The invention produces compositions having utility as pharmaceutical formulations.

### BACKGROUND

The formulation and administration of water-insoluble or sparingly water-soluble drugs is problematic because of the difficulty of achieving sufficient systemic bioavailability. Low aqueous solubility results not only in decreased bioavailability, but also in formulations that are insufficiently stable over extended storage periods. A classic example in this regard is paclitaxel, available commercially as Taxol® (Bristol-Myers Squibb). Although paclitaxel has been shown to exhibit powerful antineoplastic efficacy, its use is limited in large part by the side effects of the solvent generally used for clinical administration, a mixture of polyethoxylated castor oil and ethanol. The amount of solvent that is required to deliver an effective dose of paclitaxel is substantial, and polyethoxylated castor oil has been shown to result in serious or fatal hypersensitivity episodes in laboratory animals. Thus, extensive research has been conducted with the aim of producing an improved paclitaxel formulation having reduced toxicity. In particular, efforts have been directed toward modifying the chemistry of the drug itself to make it more hydrophilic and combining the drug with agents that produce water-soluble dispersions. Chemically modified paclitaxel analogs include sulfonated paclitaxel derivatives, amino acid esters as well as covalent conjugates of paclitaxel and polyethylene glycol (U.S. Pat. No. 5,648,506 to Desai et al.; Liu et al. (1999) J. Polymer Sci., Part A-Polymer Chem. 37:3492-3503). For the most part, however, research has focused on the entrapment of the drug in vesicles or liposomes, and on the incorporation of surfactants into paclitaxel formulations.

Liposomes are useful particles for delivering drugs that have low aqueous solubility, and representative liposomal drug delivery systems are described in U.S. Pat. No. 5,395,619 to Zalipsky et al., U.S. Pat. No. 5,340,588 to Domb, and U.S. Pat. No. 5,154,930 to Popescu et al. Liposomes are vesicles comprised of concentrically ordered lipid bilayers that encapsulate an aqueous phase. Liposomes form when phospholipids, amphipathic compounds having a polar (hydrophilic) head group covalently bound to a long-chain aliphatic (hydrophobic) tail, are exposed to water. That is, in an aqueous medium, phospholipids aggregate to form a structure in which the long-chain aliphatic tails are sequestered within the interior of a shell formed by the polar head groups. Unfortunately, use of liposomes for delivering many drugs has proven unsatisfactory, in part because liposome compositions are, as a general rule, rapidly cleared from the bloodstream. Finally, even when satisfactory liposomal formulations are prepared, it is often necessary to use some sort of physical release mechanism so that the vesicle releases the active agent in the body before it is taken up by the liver and spleen.

Numerous other methods and components have been developed for making specialized drug formulations for delivering drugs having low aqueous solubility, as well as for improving the in vivo half life or bioavailability of drugs. Some of these formulations are particulate in nature, such as micrometer or nanometer sized particles. Examples include formulations for aerosol or powder delivery of drugs to the lungs which have an optimal size of 1-5 microns. Encasement of paclitaxel microcrystals in shells of biocompatible polymeric materials has been described in U.S. Pat. No. 6,096,331 to Desai et al. However, as crystals of hydrophobic drugs may be difficult to dissolve, the rate of drug release in these formulations remains hard to control.

Incorporation of surfactants into paclitaxel formulations as described, for example, in WO 97/30695 to Yiv et al., is also problematic. Surfactants tend to alter the chemistry of a pharmaceutical formulation such that the effective ratio of drug to inactive ingredients is lowered, resulting in the need to increase dosage volume and/or administration time. Additionally, formulations that employ surfactants readily dissociate upon dilution, e.g., following intravenous injection, resulting in premature drug release. Also, many surfactants are considered unsuitable for parenteral drug administration because of their interaction with cellular membranes.

A more recent development in this area is described in WO 01/49268 to Unger et al., which describes pharmaceutical formulations that are suitable for administration of water-insoluble or sparingly water-soluble drugs such as paclitaxel, where the formulation is optimized such that the amount of drug administered is maximized while undesirable side effects are minimized, the rate of drug release can be precisely controlled, no surfactants are necessary, no liposomes or other vesicles are required, and the formulation displays excellent stability over extended storage periods.

However, in spite of the advancements made in the art regarding formulations suitable for use in delivering drugs having low aqueous solubility, there continues to be problems in the manufacture of such formulations. For example, methods to make particulate formulations are often complex and difficult to do in a sterile environment. These manufacturing processes often include a terminal energy requiring step to produce the desired small particle size. The size reducing process can typically involve spray drying (or other solvent evaporation), ball milling or microfluidization (or other similar mechanical means of size reduction), or extrusion (useful for liposome manufacturing). These processes are difficult to carry out in a sterile environment. Standard terminal filter sterilization of a final particulate formulation is difficult as the particles clog the filters.

Therefore, there remains a need in the art for improved methods of making such formulations that are simple, avoid the use of surfactants, avoid the need for high-energy terminal steps, and that can produce a sterile product. The instant invention addresses those needs.

### SUMMARY OF THE INVENTION

One aspect of the invention relates to a method of producing a sterile pharmaceutical formulation comprising: (a) admixing, in an organic solvent, a drug, and a stabilizing agent that stabilizes the drug but does not covalently bind thereto; where the organic solvent has a freezing temperature with the range of about 0-25° C.; (b) filter sterilizing the mixture; and (c) removing the organic solvent in a manner effective to provide a dry formulation of the drug. The mixture can also contain a targeting ligand and/or an excipient.

Another aspect of the invention pertains to a method of producing a sterile pharmaceutical formulation comprising: (a) admixing, in a first solvent and a second solvent, a drug, and a stabilizing agent that stabilizes the drug but does not covalently bind thereto; where the first solvent is an organic solvent having a freezing temperature with the range of about 0-25° C.; (b) filter sterilizing the mixture; and (c) removing the first solvent and second solvent in a manner effective to provide a dry formulation of the drug. The mixture can also contain a targeting ligand and/or an excipient.

Yet another aspect of the invention relates to a method of producing a sterile pharmaceutical formulation comprising: (a) admixing, in a first solvent and a second solvent, a drug, a stabilizing agent that stabilizes the drug but does not covalently bind thereto, and a water-soluble bulking agent; where the first solvent is an organic solvent having a freezing temperature with the range of about 0-25° C.; (b) filter sterilizing the mixture; and (c) removing the first solvent and second solvent in a manner effective to provide a dry formulation of the drug. The mixture can also contain a targeting ligand and/or an excipient.

Another aspect of the invention pertains to a nanoparticulate formulation prepared according to these methods.

Yet another aspect of the invention relates to an anisotropic nanoparticle or microparticle formulation of a drug comprising one or more stabilizing agents, where the particles have a rod-like appearance and the particles are at least two times longer than they are wide.

Additional aspects, advantages, and features of the invention will be set forth in part in the description that follows, and in part will become apparent to those skilled in the art upon examination of the following, or may be learned by practice of the invention.

### BRIEF DESCRIPTION OF THE DRAWINGS

FIG. 1 is a transmission electron micrograph of a paclitaxel formulation containing four-arm poly(ethylene oxide-b-ε-caprolactone).

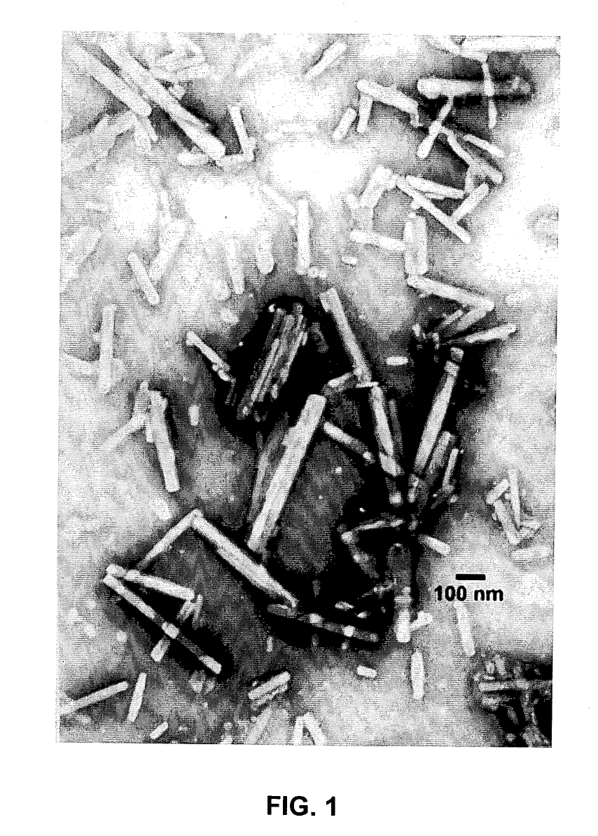

FIG. 2 is a transmission electron micrograph of an SN-38 formulation containing four-arm poly(ethylene oxide-b-ε-caprolactone), DOPG, and DOPE-PEG2000. This micrograph is representative of the formulation described in Example 8.

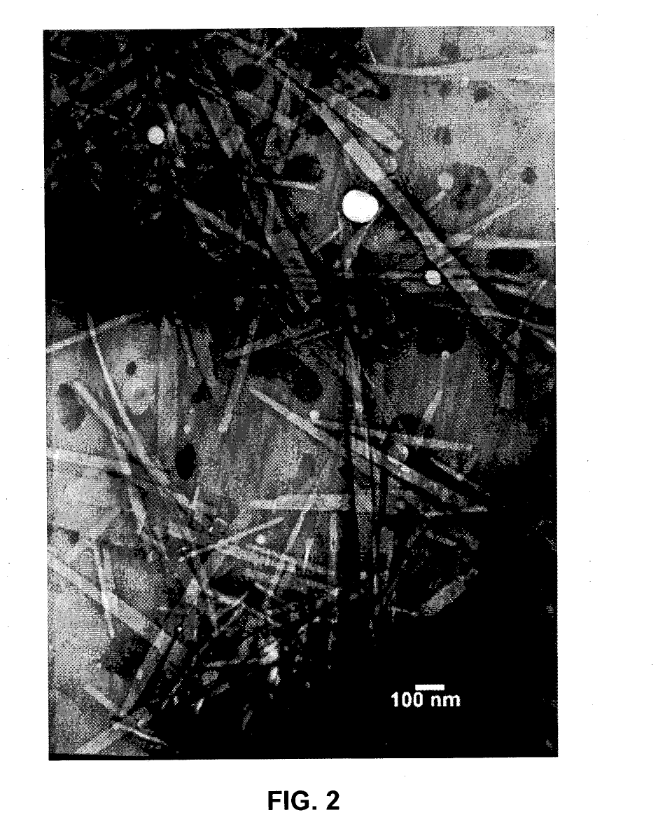

### DETAILED DESCRIPTION OF THE INVENTION

I. Definitions and Overview

It is to be understood that unless otherwise indicated, this invention is not limited to specific active agents, hydrophilic polymers, copolymers, phospholipids, excipients, methods of manufacture or the like, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “an active agent” or “a drug” in a formulation means that more than one active agent can be present, reference to “a stabilizing agent” includes combinations of stabilizing agents, reference to “a phospholipid” includes mixtures of phospholipids, and the like.

In this specification and in the claims that follow, reference will be made to a number of terms that shall be defined to have the following meanings:

By “pharmaceutically acceptable” is meant a material that is not biologically or otherwise undesirable, i.e., the material may be administered to an individual along with the selected active agent without causing any undesirable biological effects or interacting in a delete-rious manner with any of the other components of the pharmaceutical composition in which it is contained.

“Pharmaceutically or therapeutically effective dose or amount” refers to a dosage level sufficient to induce a desired biological result. That result can be alleviation of the signs, symptoms, or causes of a disease, or any other desired alteration of a biological system.

The term “treat” as in “to treat a disease” is intended to include any means of treating a disease in a mammal, including (1) preventing the disease, i.e., avoiding any clinical symptoms of the disease, (2) inhibiting the disease, that is, arresting the development or progression of clinical symptoms, and/or (3) relieving the disease, i.e., causing regression of clinical symptoms.

The terms “disease,” “disorder” and “condition” are used interchangeably herein to refer to a physiological state that may be treated using the formulations of the invention.

The terms “drug,” “active agent” and “therapeutic agent” are used interchangeably herein to refer to a chemical material or compound which, when administered to an organism (human or animal), induces a desired pharmacologic effect. Included are analogs and derivatives (including salts, esters, prodrugs, and the like) of those compounds or classes of compounds specifically mentioned which also induce the desired pharmacologic effect.

The number given as the “molecular weight” of a compound, as in the molecular weight of a hydrophilic polymer such as polyethylene glycol, refers to weight average molecular weight M_{w}.

The “solubility” of a compound refers to its solubility in the indicated liquid determined under standard conditions, e.g., at room temperature (typically about 25° C.), atmospheric pressure, and neutral pH.

“Lipid” refers to a synthetic or naturally-occurring compound which is generally amphipathic and biocompatible. The lipids typically comprise a hydrophilic component and a hydrophobic component. Exemplary lipids include, for example, fatty acids, neutral fats, phospholipids, phosphatides, glycolipids, surface-active agents, aliphatic alcohols, and steroids. Specifically, the choice of the term is used to distinguish it from the more stringently defined terms “liposome” and “micelle,” wherein a liposome implies a vesicular structure with a defined interior aqueous compartment. The arrangement of molecules in a liposome gives rise to a vesicle of at least one lamellar bilayer. Drugs may be sequestered within the interior of liposomes, embedded within the lipid matrix, or affixed to the outside surface of the liposome. In a micelle, there is an arrangement of polar amphipathic molecules, wherein the hydrophilic portion (heads) of the structure defines the exterior surface and the hydrophobic portion (tails) resides interiorly, away from the medium. A micelle is not, by definition, a bilayer, and thus its size and effective carrying capacity is limited according to properties defined by the critical micelle concentration for a given compound. In contrast to liposomes and micelles, lipidic structures are non-liposomal, non-micellar associations of lipid and drug.

The term “lecithin” refers the class of phospholipids called phosphatidylcholines, and generally refers to natural phosphatidylcholines such as dioleylphosphatidylcholine, phosphatidyl inositol and phosphatidyl choline. Such naturally occurring phospholipids are composed of phosphate, choline, glycerol (as the ester), and two fatty acids, and are exclusively modified with phosphatidylcholine at the 3-position of the glycerol. The fatty acyl moieties attached at the 1 and 2 hydroxyl positions of glycerol may be saturated, unsaturated, or a combination of both. Lecithin does not comprise anionic phospholipids such as phosphatidylglycerol, or chemically modified, synthetic phospholipids.

“Polymer” refers to molecules formed from the chemical union of two or more repeating units. Accordingly, included within the term “polymer” may be, for example, dimers, trimers and oligomers. The polymer may be synthetic, naturally-occurring or semisynthetic. In one embodiment, the term “polymer” refers to molecules which comprise 10 or more repeating units. In other embodiments, the polymers which may be incorporated in the compositions described herein contain no denatured naturally occurring proteins that are crosslinked by disulfide linkages.

“Covalent association” refers to an intermolecular association or bond which involves the sharing of electrons in the bonding orbitals of two atoms.

“Non-covalent association” refers to intermolecular interaction among two or more separate molecules which does not involve a covalent bond. Intermolecular interaction is dependent upon a variety of factors, including, for example, the polarity of the involved molecules, the charge (positive or negative), if any, of the involved molecules, and the like. Non-covalent associations are preferably selected from the group consisting of ionic interaction, dipole-dipole interaction and van der Waal's forces and combinations thereof.

“Targeting ligand” refers to any material or substance which may promote targeting of tissues and/or receptors in vivo with the compositions described herein. The targeting ligand may be synthetic, semi-synthetic, or naturally-occurring. Materials or substances which may serve as targeting ligands include, for example, proteins, including antibodies, glycoproteins and lectins, peptides, polypeptides, saccharides, including mono- and polysaccharides, vitamins, steroids, steroid analogs, hormones, cofactors, bioactive agents, prostacyclin and prostaglandin analogs, and genetic material, including nucleosides, nucleotides and polynucleotides.

“Peptide” or “polypeptide” refer to nitrogenous polymeric compounds which may contain from about 2 to about 100 amino acid residues. In certain embodiments, the peptides which may be incorporated in the compositions described herein contain no denatured naturally occurring proteins that are crosslinked by disulfide linkages.

“Protein” refers to a nitrogenous polymer compound which may contain more than about 100 amino acid residues. In certain embodiments, the proteins which may be incorporated in the compositions described herein contain no denatured naturally occurring proteins that are crosslinked by disulfide linkages.

“Nanoparticles” are defined strictly according to size in that they have diameters less than one micrometer. The term may embrace amorphous, structured, or partially crystalline forms. “Nanocrystals” by contrast are defined as structures with sizes less than one micrometer, but that have at least 99% crystalline structure, regardless of whether the molecular composition of said crystal is purely one component, e.g., drug, or drug in close association with another component.

The term “stabilizer” refers to materials such as lipids, polymers, polymer-lipid conjugates, and other coating agents, surfactants, or compounds that alter the physical and chemical properties affecting aqueous solubility of a drug when placed in a noncovalent admixture with the drug or drugs.

“Optional” or “optionally” means that the subsequently described circumstance may or may not occur, so that the description includes instances where the circumstance occurs and instances where it does not.

II. Manufacture and Storage

The present invention is based on the formation of a self-assembling noncovalent complex of drug molecules with a polymer, lipid, or conjugated polymer-lipid, stabilizing agent. This drug/stabilizer complex allows for the formation of an aqueous suspension of nanoparticles of the complex without requiring chemical modification of the drug. This technology can be applied to many drugs having poor solubility in water, e.g., camptothecin, paclitaxel, and so forth. For example, problems related to stability, toxicity of the carrier, and large injection volume of currently available formulations of paclitaxel are well documented. Nanoparticle solubilization technology enables the preparation of drug formulations with decreased toxicity and improved efficacy.

The invention relates to a method of producing a unique class of particles, generally nanoparticles, ranging in size from about 10 nm to 1,000 nm, preferably about 100 nm to 900 nm, more preferably about 200 mm to 800 nm. The resulting nanoparticles are biocompatible and highly useful for drug delivery. In one embodiment of the invention, the methods described herein produce an anisotropic nanoparticle or microparticle formulation of a drug comprising one or more stabilizing agents, where the particles (i.e., nanoparticles or microparticles) have a rod-like appearance and the particles are at least two times longer than they are wide.

The methods of the invention provide for the manufacture of a sterile formulation using minimal steps, where sterilization is done prior to nanoparticle formation. One advantage of this method is that it avoids the need for a terminal energy-requiring step that involves the application of mechanical shear stress to achieve a finely ground powder of nanoparticulate material. Examples of such terminal energy-requiring steps include microfluidization, sonication, extrusion, ball milling, homogenization, and the like. While such methods are capable of producing the desirable nanoparticles, it is extremely difficult to conduct such processes under sterile conditions and it is equally difficult to sterilize the nanoparticles produced by such processes.

In one embodiment of the method of the invention, a sterile pharmaceutical formulation is produced by admixing, in an organic solvent, a drug, and a stabilizing agent that stabilizes the drug but does not covalently bind thereto; wherein the organic solvent has a freezing temperature with the range of about 0-25° C. The mixture can also contain a targeting ligand and/or an excipient. The mixture is then filter sterilized. The solvent is then removed in a manner effective to provide a dry formulation of the drug. The filter sterilizing step is therefore done prior to filling into sterile vials and lyophilizing the formulation to remove the solvent.

An exemplary protocol is as follows. The drug, stabilizing agent(s), and any other additives (e.g., targeting ligand, excipient, etc.) are weighed out into a suitable container. These ingredients are then dissolved in the primary solvent, e.g., t-butanol, alone or in combination with a second solvent, e.g., 5% DMSO/95% t-butanol with or without water. The resulting solution is then filtered through a 0.2 micron solvent-safe filter directly into sterile vials in an aseptic suite. The vials are then frozen and subjected to a lyophilization cycle. The vials containing dry powder are then sealed to preserve the sterility of the product.

Another exemplary protocol involves dissolving the stabilizing agent(s), and any other additives in the primary solvent, and optionally heating. The drug is dissolved in a secondary solvent separately. The dissolved drug is then added directly to the primary solvent solution, sterile filtered into an aseptic suite, and aliquoted into sterile vials.

A. Mixing

In one embodiment, the drug, stabilizing agent and any other additives (e.g., targeting ligand, excipients, and so forth) are mixed with an organic solvent having a freezing temperature with the range of about 0-25° C. (also referred to as the “primary solvent”). In another embodiment, the stabilizing agent, therapeutic agent, and any other additives are mixed with the primary solvent and a secondary solvent. In another embodiment, the stabilizing agent, therapeutic agent, and any other additives are mixed with the primary solvent, a secondary solvent and a water-soluble bulking agent. After mixing and sterile filtration, the solvents are removed in a manner effective to provide a dry formulation of the drug.

The mixing step can involve mixing all ingredients together in one solution or may be accomplished by mixing one or more ingredients separately then combining the resulting solutions prior to the solvent removal step.

The mixing step(s) may be conducted at room temperature or gently heated in order to assist in dissolving the drug and any additives into the primary solvent (and secondary solvent if included). The appropriate temperature to heat any such solution in order to completely dissolve the ingredients would readily be determined by those skilled in the art.

B. Filter Sterilization

Since the particles produced by the method are not formed until hydration of the final, lyophilized product, the formulation can be sterilized with a sterilizing filter prior to lyophilization.

Typically, this will involve filtering the solution of the organic solvent and the drug and formulation ingredients using a 0.2 micron filter that is solvent compatible, to make a sterile solution. The sterile solution is then aliquoted directly into dose-sized sterile vials or may be aliquoted at a later time, such as in a sterile fill.

C. Solvent Removal

The sterile drug/solvent solution is then frozen and the solvent removed in a manner effective to provide a dry formulation of the drug, e.g., the solution can be lyophilized. The dry formulation can be stored and/or hydrated with a suitable sterile diluent prior to use.

The solvent may also be removed by lyophilization, by spray drying or by subjecting the mixture to rotary evaporation to yield a powder. When the solvent is removed by rotary evaporation, an agglomerated intermediate product is produced, which is then deagglomerated to provide the dry formulation of the drug.

Alternatively the components of the final product may be dissolved in a supercritical fluid such as compressed carbon dioxide, and then ejected under pressure and shearing force to form dried particles of the drug-containing formulation.

A suitable lyophilization cycle can be readily determined by those skilled in the art, as lyophilization conditions may vary. For example, primary drying conditions may vary from −50° C. to −5° C. The length of the cycle is generally known to those skilled in the art, for example, the cycle length may vary from 8 to 48 hours, generally, sufficient time to remove the solvent or liquid from the product. The secondary drying conditions may vary from 0° C. to 50° C.

In a preferred embodiment, the formulation is stored in lyophilized form since the lyophilized product may be stored for long periods of time.

The lyophilized product may be used without hydration for some types of administration, such as pulmonary dry powder, tablets, capsules and the like, or administration via the nasal route. For administration in aqueous solution, the lyophilized product may be rehydrated prior to use by mixing in a suitable aqueous pharmaceutically acceptable carrier, which is typically an aqueous liquid (e.g., water, isotonic saline solution, lactated Ringer's solution, 5% dextrose, buffered solution such as a citrate or phosphate buffer, etc.). Typically, the rehydrated product will have a total solute concentration in the range of about 50 to 100 mg/ml and a drug concentration in the range of about 1 to 20 mg/ml, preferably about 5 to 15 mg/ml. The rehydrated formulation may be stored in this aqueous state, e.g., in pre-filled syringes or vials, prior to use. The rehydrated product can also be sonicated as described above, without compromising sterility.

The lyophilized and rehydrated formulations may be stored at various temperatures such as freezing conditions (below about 0° C. and as low as about −40° C. to −100° C.), refrigerated conditions generally between about 0° C. and 15° C., room temperature conditions generally between about 15° C. and 28° C., or at elevated temperatures as high as about 40° C.

The particle size of individual particles within the formulation will vary, depending upon the molecular weight and concentration of the hydrophilic polymer, the amount of drug as well as its solubility profile (i.e., its solubility in water and the hydrophilic polymer), the use of stabilizing agents, and the conditions used in manufacturing. That is, stabilizing agents and various excipients may be used to facilitate rehydration and provide a substantially homogeneous dispersion.

As noted above, the particles produced by the methods of the invention will typically range in size from about 10 nm to 10,000 mm, preferably about 50 nm to 1,000 nm, more preferably about 200 nm to 800 nm (the values given are the number weighted average). Other moieties may be incorporated into the present formulations as excipients in order to reduce the particle size of the stabilized drug matrix to tailor the formulation for its intended use. For example, small particles, by virtue of their larger accessible surface-to-volume ratio, tend to release drug quite rapidly, while larger particles, will provide for far more gradual, sustained release of drug. For pulmonary administration, particle size is optimally within the range of about 500 to 5,000 nm. For intramuscular and subcutaneous injection, particle size should be in the range of about 1 nm to 10,000 nm. For intravenous administration, particle size is optionally in the range of about 10 nm to 1,000 nm, preferably about 30 to 250 nm. For interstitial administration and fracture or wound packing, and for embolization, particle size can be up to 10,000 nm.

D. Optional Steps

The hydrated lyophilized powder may be sonicated to further reduce particle size and facilitate dissolution. Sonicating the product in the sealed vial using a water bath sonicator will not impact the sterility of the product, and those skilled in the art will recognize there are many sonicating methods generally available.

III. Method Materials

A. Organic Solvent

Suitable organic solvents are those that are miscible or co-miscible with the formulation components (drug, stabilizing agent, optional targeting ligand, and optional excipient), and has a freezing temperature with the range of about 0-25° C. Exemplary organic solvents include, by way of example and not limitation, tert-butyl alcohol (t-butanol), cyclohexane, dimethyl carbonate, dimethyl sulfoxide, and acetic acid.

B. Secondary Solvent

The method may also involve adding a second solvent, which is miscible or co-miscible with the first organic solvent, and can be an aqueous or an organic solvent. The second solvent is preferably selected so as to decrease the polarity of the first organic solvent, and is also preferably selected so that all ingredients of the formulation are soluble therein.

Exemplary secondary solvents include, by way of example and not limitation, alkylated alcohols, ethers, acetone, alkanes, dimethyl sulfoxide (DMSO), chloroform, cyclic hydrocarbons, toluene, benzene, N,N-dimethylformamide (DMF), and mixtures thereof such as a benzene/methanol solvent system. Exemplary ethers include methoxylated ethers, alkylated ethers, diether, triethers, oligo ethers, polyethers, cyclic ethers, and crown ethers. Exemplary alkylated alcohols include methanol, ethanol and isopropanol. Exemplary alkanes include hexane. In one embodiment, water can then added in as a third solvent.

Other suitable secondary solvents are known to those skilled in the art of pharmaceutical formulation and drug delivery and/or described in the pertinent texts and literature. See Remington: The Science and Practice of Pharmacy, 19th Ed. (Easton, Pa.: Mack Publishing Co., 1995), which discloses conventional methods of preparing pharmaceutical compositions that may be used as described or modified to prepare pharmaceutical formulations of the invention. In addition, any solvents identified by the US Food and Drug Administration as class II and class III solvents can also be used.

C. Water Soluble Bulking Agents

The method may also involve adding a water-soluble bulking agent. The water-soluble bulking agent is typically added as an aqueous solution. The water-soluble bulking agent also functions as a cryoprotectant. Such agents include, by way of example and not limitation, sorbitol, mannitol, xylitol, hydrogenated starch hydrolysates, maltitol, lactitol, maltitol, hydrogenated isomaltulose, erythritol, inositol, sucrose, and trehalose.

IV. Formulation Materials

The pharmaceutical formulations of the invention are advantageously used to deliver any drug whose systemic bioavailability (including oral bioavailability) can be enhanced by increasing the solubility of the drug in water. Thus, the drugs that are preferred for use in conjunction with the present invention are generally hydrophobic in nature, tending toward low aqueous solubility. The invention provides a method of incorporating such drugs in a composition comprised of a matrix of a stabilizing agent that physically entraps and thereby immobilizes the drug, but does not covalently bind thereto.

A. The Stabilizing Agent

The stabilizing agents of the present invention are polymers, lipids, polymer-lipid conjugates and mixtures thereof, that are capable of forming noncovalent complexes with the drug of interest. The stabilizing agent used in the methods of the invention is spatially stabilized so as to facilitate physical entrapment and thus immobilization of the active agent; that is, the “spatially stabilized” stabilizing agent forms a matrix or three-dimensional structure in which discrete regions of drug are dispersed. Any material that can form such a matrix can be used in conjunction with the invention, providing that the material is sufficiently hydrophilic to increase the aqueous solubility of the entrapped drug.

In one embodiment, the stabilizing agent contains a mixture of polymeric stabilizing agents and lipid stabilizing agents. For example, a particularly preferred formulation might contain about 2 parts drug such as SN-38, 1 part poloxamer, and 8 parts by weight of phosphatidylglycerol. Preferred ranges for the ratio of drug to lipid to polymeric component when combinations of stabilizing agents are used range between approximately 1:2:1 to approximately 1:20:5, most preferably from approximately 1:5:1 to approximately 1:10:2. In embodiments where lipids and polymers are both used as the stabilizing agent, the polymeric component of the stabilizing agent is generally added during rehydration

1. Polymer Stabilizing Agents

Suitable polymer stabilizing agents can be hydrophilic and/or hydrophobic polymers, with hydrophilic polymers being preferred. The term “hydrophilic,” as used herein, refers to a composition, substance or material, for example, a polymer, which may generally readily associate with water. Thus, although the hydrophilic polymers that may be employed in the present invention may have domains of varying type, for example, domains which are more hydrophilic and domains which are more hydrophobic, the overall nature of the hydrophilic polymers is preferably hydrophilic, it being understood, of course, that this hydrophilicity may vary across a continuum from relatively more hydrophilic to relatively less hydrophilic. The term “hydrophobic,” as used herein, refers to a composition, substance or material, for example, a polymer, which generally does not readily associate with water. Thus, although the hydrophobic polymers that may be employed may have domains of varying type, for example, domains which are more hydrophobic and domains which are more hydrophilic, the overall nature of the hydrophobic polymers is preferably hydrophobic, it being understood, of course, that this hydrophobicity may vary across a continuum from relatively more hydrophobic to relatively less hydrophobic.

The polymers can be linear or branched structures, including block copolymers and branched block copolymers. It should be understood that the term “branched,” when applied to polymers, also includes any dendritic, star, or star-like polymer. In some embodiments, the present polymers may be in the form of a matrix or three-dimensional structure which may be spatially stabilized. The term “matrix,” as used herein, refers to a three dimensional structure which may comprise, for example, a single molecule of a polymer, such as PEG associated with one or more molecules of a therapeutic agent, or a complex comprising a plurality of polymer molecules in association with a therapeutic agent. The morphology of the matrix may be, for example, particulate, where the particles are preferably in the form of nanoparticulate structures, or the morphology of the matrix may be micellar. The term “spatially stabilized,” as used herein, means that the relative orientation of an active agent, when present in the matrices of the present invention, may be fixed or substantially fixed in three-dimensional space, without directional specification. Thus, compositions described herein may facilitate physical entrapment and, preferably, immobilization or substantial immobilization, of one or more active agents. Generally, although not necessarily, the spatially stabilized matrix may be sterically constrained. In one embodiment, the matrices are hydrophilic, i.e., the overall nature of the matrices is hydrophilic.

Stability may be evaluated, for example, by placing the pharmaceutical composition in water, and monitoring for dissolution and/or release of the therapeutic agent. Preferably, the present pharmaceutical compositions may be spatially stable for at least about 5 minutes, more preferably at least about 30 minutes, even more preferably for more than an hour. In certain embodiments, the pharmaceutical composition may be spatially stable in solution for days, weeks, and even months.

In certain embodiments, the present matrices may comprise a network of particulate structures. The size and shape of the particulate structures may vary depending, for example, on the particular polymer employed, the desired rate of release of the therapeutic agent, and the like. For example, the particulate structures may be spherical in shape, or they may take on a variety of regular or irregular shapes. With regard to the size of the particles, in one embodiment, the diameter of the particles may range from about 1 nanometer (nm) to less than about 1000 nm, and all combinations and subcombinations of ranges and specific particle sizes therein.

A wide variety of polymers may be employed in the present compositions and formulations. Generally speaking, the polymer is one which has the desired hydrophilicity and/or hydrophobicity, and which may form matrices, as well as covalent attachments with targeting ligands, as described in detail herein. The polymer may be crosslinked or non-crosslinked, with substantially non-crosslinked polymers being preferred. The terms “crosslink,” “crosslinked,” and “crosslinking,” as used herein, generally refer to the linking of two or more compounds or materials, for example, polymers, by one or more bridges. The bridges, which may be composed of one or more elements, groups or compounds, generally serve to join an atom from a first compound or material molecule to an atom of a second compound or material molecule. The crosslink bridges may involve covalent and/or non-covalent associations. Any of a variety of elements, groups and/or compounds may form the bridges in the crosslinks, and the compounds or materials may be crosslinked naturally or through synthetic means. For example, crosslinking may occur in nature in materials formulated from peptide chains which are joined by disulfide bonds of cystine residues, as in keratins, insulin, and other proteins. Alternatively, crosslinking may be effected by suitable chemical modification, such as, for example, by combining a compound or material, such as a polymer, and a chemical substance that may serve as a crosslinking agent, which are caused to react, for example, by exposure to heat, high-energy radiation, ultrasonic radiation, and the like. Examples include, for example, crosslinking with sulfur which may be present, for example, as sulfhydryl groups in cysteine residues, to provide disulfide linkages, crosslinking with organic peroxides, crosslinking of unsaturated materials by means of high-energy radiation, crosslinking with dimethylol carbamate, and the like. The term “substantially,” as used in reference to crosslinking, means that greater than about 50% of the involved compounds or materials contain crosslinking bridges. In certain embodiments, greater than about 60% of the compounds or materials contain crosslinking bridges, with greater than about 70% being a preferred embodiment. Even more preferably, greater than about 80% of the compounds or materials contain crosslinking bridges, with greater than about 90% being still more preferred. In certain embodiments, greater than about 95% of the compounds or materials contain crosslinking bridges. If desired, the substantially crosslinked compounds or materials may be completely crosslinked (i.e., about 100% of the compounds or materials contain crosslinking bridges). In other embodiments, the compounds or materials may be substantially (including completely) non-crosslinked. The term “substantially,” as used in reference to non-crosslinked compounds or materials, means that greater than about 50% of the compounds or materials are devoid of crosslinking bridges. In a preferred embodiment, greater than about 60% of the compounds or materials are devoid of crosslinking bridges, with greater than about 70% being more preferred. Even more preferably, greater than about 80% of the compounds or materials are devoid of crosslinking bridges, with greater than about 90% being still more preferred. In particularly preferred embodiments, greater than about 95% of the compounds or materials are devoid of crosslinking bridges. If desired, the substantially non-crosslinked compounds or materials may be completely non-crosslinked (i.e., about 100% of the compounds or materials are devoid of crosslinking bridges).

Examples of suitable polymeric stabilizing agents include, but are not limited to, polyethylene glycol, polypropylene glycol, polyvinyl alcohol, polyvinyl pyrrolidone, polylactide, poly(lactide-co-glycolide), polysorbate, polyethylene oxide, polycaprolactone, polypropylene oxide, poly(ethylene oxide-co-propylene oxide), poly(oxyethylated) glycerol, poly(oxyethylated) sorbitol, poly(oxyethylated) glucose), and derivatives, mixtures, and copolymers thereof. Examples of suitable derivatives include those in which one or more C—H bonds, e.g., in alkylene linking groups, are replaced with C—F bonds, such that the polymers are fluorinated or even perfluorinated.

In one embodiment, the polymer comprises repeating alkylene units, wherein each alkylene unit optionally contains from one to three heteroatoms selected from —O—, —N(R)— or —S(O)ₙ—, where R is hydrogen or alkyl and n is 0, 1 or 2. Preferably, the number of alkylene units are 2, 3, 4, or 5 units. The polymers may be linear (e.g., the type AB random sequence of units or AB block where two or more units of A are linked to two or more units of B, type ABA, ABABA or ABCBA alternating units or blocks, and the like), branched (e.g., the type AₙB or BAₙC, and the like, where A is at least n-valent, and n is an integer ranging from about 3 to about 50, and all combinations and subcombinations of ranges and specific integers therein or multiple A's extending from one B), with branched polymers being preferred. When a branched polymer is employed, particularly when the branched polymer includes an inner, more hydrophobic core region and an outer, more hydrophilic region, the resulting delivery system may be in the form of a nanoparticle. An exemplary illustration of such a delivery system occurs when a branched block copolymer structure binds a plurality of molecules of an active agent, for example, SN-38. In another embodiment, the branched polymer used includes an inner more hydrophilic core region and an outer, more hydrophobic region, the resulting delivery system is in the form of a nanoparticle. Once again, this branched block copolymer binds a plurality of molecules of an active agent, for example, SN-38. When branched polymers are used, they contain between about 4 and 40 arms, more preferably between 4 and 10 arms, more preferably between 4 and 8 arms, and most preferably 4 arms. When branched polymers are used, these preferably contain but are not limited to one or a combination of two or more of the following polymers; polyethylene glycol, polypropylene glycol, polycaprolactone, polylactide, polyglycolide, and, polylactide-co-glycolide.

Particularly useful polymers for stabilizing the nanoparticles include linear or branched polyethylene glycol (PEG), and copolymers of PEG with polypropylene oxide, such as the PLURONICS® (BASF Corporation, Mount Olive, N.Y.). Linear block polymers are poloxamer, a block copolymer of propylene oxide flanked on each end by ethylene oxide; and poloxamine, a polyalkoxylated symmetrical block polymer of ethylene diamine conforming to the general type [(PEG)_{X}—(PPG)_{Y}]₂-NCH₂CH₂N-[(PPG)_{Y}-(PEG)_{X}]₂. Preferred species of poloxamer are the PLURONICS® with PLURONIC® F68 being highly preferred. Suitable poloxamines include the TETRONICS® with TETRONIC® 908 as a preferred species with a molecular weight of 25,000 daltons. Other derivatives with shorter PEG and PPG copolymeric chains having molecular weights between 1650 daltons to 25 kilodaltons are also suitable. Branched block copolymers are especially useful as stabilizing agents, particularly those with a molecular weight of 8000 to 15000 Daltons containing both hydrophilic and hydrophobic blocks. These branched block copolymers may be comprised of either a hydrophobic core and hydrophilic distal arms, or a hydrophilic core and hydrophobic distal arms.

As noted above, a preferred polymer for use in the present formulations is polyethylene glycol (PEG) or a copolymer thereof, e.g., polyethylene glycol containing some fraction of other monomer units (e.g., other alkylene oxide segments such as propylene oxide), with polyethylene glycol itself most preferred. The polyethylene glycol used may be either linear or a branched PEG. In certain embodiments, the polymer may be covalently associated with a lipid, such as a phospholipid moiety in which the hydrophobic chains of the phospholipids may tend to associate in an aqueous medium. Combinations of different types of PEG (e.g., branched PEG and linear PEG, star PEG and linear PEG, branched PEG and phospholipid-conjugated linear PEG, etc.) may also be employed. In other embodiments the polymer may be covalently associated with a fatty acid with a carbon chain length of 6 to 22 carbons.

With respect to branched polymers, the molecular weight of the entire branched polymer may range from about 2000 to 1,000,000 daltons, preferably from about 5000 to 100,000 daltons, more preferably from about 10,000 to 60,000 daltons, and still more preferably about 20,000 daltons. Preferably, each arm has the same unit size of polymer, such as PEG, e.g., about 2500 daltons each for an 8-armed PEG. In the case of a branched copolymer, the various percentages of the hydrophobic and hydrophilic monomers or blocks in each arm may vary. For example, with an 8 arm branched copolymer of polypropylene glycol (PPG) and PEG, when 50% is PPG and 50% is PEG, both the PPG segment and the PEG segment will have a molecular weight about 1250 daltons, with the PEG forming the outer portion of the arm.

Branched PEG molecules will generally although not necessarily have a molecular weight in the range of approximately 1,000 to 600,000 daltons, more typically in the range of approximately 2,000 to 100,000 daltons, preferably in the range of approximately 5,000 to 40,000 daltons. Branched PEG is commercially available, such as from Nippon Oil and Fat (NOF Corporation, Tokyo, Japan) and from Shearwater Polymers (Huntsville, Ala.), or may be readily synthesized by polymerizing lower molecular weight linear PEG molecules (i.e., PEG 2000 or smaller) functionalized at one or both termini with a reactive group. For example, branched PEG can be synthesized by solution polymerization of lower molecular weight PEG acrylates (i.e., PEG molecules in which a terminal hydroxyl group is replaced by an acrylate functionality —O—(CO)—CH═CH₂) or methacrylates (similarly, PEG molecules in which a hydroxyl group is replaced by a methacrylate functionality —O—(CO)—C(CH₃)≡CH₂) in the presence of a free radical polymerization initiator such as 2,2′-azobisisobutyronitrile (AIBN). If desired, mixtures of PEG monoacrylates or monomethacrylates having different molecular weights can be used in order to synthesize a branched polymer having “branches” or “arms” of differing lengths. Branched PEGs have 2 or more arms but may have as many as 1,000 arms. The branched PEGs herein preferably have about 4 to 40 arms, more preferably about 4 to 10 arms, and most preferably about 4 to 8 arms. Higher molecular weight, highly branched PEG, e.g., branched PEG having a molecular weight of greater than about 10,000 and at least about 1 arm (i.e., one branch point) per 5,000 daltons, will sometimes be referred to herein as “dendrimeric” PEG. Dendrimeric PEG may preferably be formed by reaction of a hydroxyl-substituted amine, such as triethanolamine, with lower molecular weight PEG that may be linear, branched or star, to form a molecular lattice that may serve as the spatially stabilized matrix for delivery of an entrapped active agent. Dendrimeric structures, including dendrimeric PEG, are described, inter alia, by Liu et al. (1999) PSTT 2(10):393-401.

Star molecules of PEG are available commercially (e.g., from Shearwater Polymers, Huntsville, Ala.) or may be readily synthesized using free radical polymerization techniques as described, for example, by Gnanou et al. (1988) Makromol. Chem. 189:2885-2892 and U.S. Pat. No. 5,648,506 to Desai et al., the disclosures of which are hereby incorporated herein by reference, in their entireties. Star PEG typically has a central core of pentaerythritol or glycerol. Preferred molecular weights for star molecules of PEG may be from about 1000 to 500,000 Daltons, with molecular weights of about 10,000 to 200,000 being preferred. The therapeutic agent may be associated with the branches and/or arms of the matrix, and/or may be associated with the core portions of the matrix structures.

The polymers employed in the present matrices may be selected so as to achieve the desired chemical environment to which the therapeutic agent may be exposed. Specifically, in the case, for example, of star polymers, the inner core region may generally be relatively more hydrophobic, and the arms or branches may generally be more hydrophilic. Alternatively, the inner core region may generally be relatively more hydrophilic, and the arms or branches may generally be more hydrophobic. It should be understood, however, that the chemical structures of the core, arms and branches of the polymer may be selected, as desired, so as to modify or alter the generally hydrophobic nature of the core (for example, by increasing or decreasing the core's hydrophobicity) and the generally hydrophilic nature of the arms and/or branches (for example, by increasing or decreasing the hydrophilicity of the arms and/or branches).

The number of “branches” or “arms” in star polymers may range from about 3 to 50, with from about 3 to 30 being preferred, and from about 3 to 12 branches or arms being more preferred. Even more preferably, the star polymers contain from about 4 to 8 branches or arms, with either about 4 arms or about 8 arms being still more preferred, and about 4 arms being particularly preferred. Preferred branched polymers may contain from about 3 to 1000 branches or arms (and all combinations and subcombinations of ranges and specific numbers of branches or arms therein). As noted above, preferred branched polymers may have from about 4 to 40 branches or arms, even more preferably from about 4 to 10 branches or arms, and still more preferably from about 4 to 8 branches or arms.

In accordance with certain preferred embodiments, the polymer, whether linear, star or branched, may be selected from the group consisting of polyalkylene oxides, polyalkyleneimines, polyalkylene amines, polyalkene sulfides, polyalkylene sulfonates, polyalkylene sulfones, poly(alkylenesulfonylalkyleneimine)s, polycaprolactones, polylactides, polyglycolides, and derivatives, mixtures and copolymers thereof.

The polymer may also be modified in one or more ways. For drugs that are ionized at physiological pH, charged groups may be inserted into the hydrophilic polymer in order to modify the sustained release profile of the formulation. To reduce the rate of drug release and thereby provide sustained delivery over a longer time period, negatively charged groups such as phosphates and carboxylates are used for cationic drugs, while positively charged groups such as quaternary ammonium groups are used in combination with anionic drugs. To insert such groups, a terminal hydroxyl group of a hydrophilic polymer such as PEG may be converted to a carboxylic acid or phosphate moiety by using a mild oxidizing agent such as chromic (VI) acid, nitric acid or potassium permanganate. A preferred oxidizing agent is molecular oxygen used in conjunction with a platinum catalyst. Introduction of phosphate groups may be carried out using a phosphorylating reagent such as phosphorous oxychloride (POCl₃). Terminal quaternary ammonium salts may be synthesized, for example, by reaction with a moiety such as

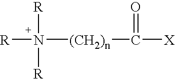

wherein R is H or lower alkyl (e.g., methyl or ethyl), n is typically 1 to 4, and X is an activating group such as Br, Cl, I or an —NHS ester. If desired, such charged polymers may be used to form higher molecular weight aggregates by reaction with a polyvalent counter ion.

Other possible modifications to the hydrophilic polymer include, but are not limited to, the following. A terminal hydroxyl group of a PEG molecule may be replaced by a thiol group using conventional means, e.g., reacting hydroxyl-containing PEG with a sulfur-containing amino acid such as cysteine, using a protected and activated amino acid. The resulting polymer (“PEG-SH”) is also commercially available, for example from Shearwater Polymers. Alternatively, a mono(lower alkoxy)-substituted PEG such as monomethoxy polyethylene glycol (MPEG) may be used instead of polyethylene glycol per se, so that the polymer terminates with a lower alkoxy substituent (such as a methoxy group) rather than with a hydroxyl group. Similarly, an amino substituted polymer such as PEG amine, may be used in lieu of the corresponding non-substituted polymer, e.g., PEG, so that a terminal amine moiety (—NH₂) may be present rather than a terminal hydroxyl group.

In addition, as discussed above, the polymer may contain two or more types of monomers, as in a copolymer wherein propylene oxide (—CH₂CH₂CH₂O—), lactide (—OCH(CH₃)CO—), glycolide (—OCH₂CO—), or caprolactone groups (—O(CH₂)₅CO—), have been substituted for some fraction of ethylene oxide groups (—CH₂CH₂O—) in polyethylene glycol, for example, four-arm poly(ethylene oxide-b-lactide) L form or four-arm poly(ethylene oxide-b-α-caprolactone) (“branched PEG-b-polycaprolactone”). Incorporating these groups may tend to increase the stability of the spatially stabilized matrix that entraps the drug, thus decreasing the rate at which the drug may be released in the body. The more hydrophobic the drug and the larger the fraction of propylene oxide or other hydrophobic blocks, the slower the drug release rate will be. Generally speaking therefore, by increasing the hydrophobicity of the camptothecin analog complex and the fraction of hydrophobic blocks may result in a slower rate of release of the agent from the matrix.

Other suitable PEG copolymers may be synthesized from polymerizable aldehydes that optionally contain additives and/or crosslinking elements capable of copolymerization, surfactants or surfactant mixtures, coupling agents, biomolecules or macromolecules bound by these coupling agents, as well as diagnostically or therapeutically effective components.

The monomers encompassed herein include, but are not limited to, alpha/beta-unsaturated aldehydes, e.g., acrolein, crotonaldehyde, propionaldehyde, alpha-substituted acrolein derivatives, e.g., alpha-methyl acrolein, alpha-chloroacrolein, alpha-phenyl acrolein, alpha-ethyl acrolein, alpha-isopropyl acrolein, alpha-n-butyl acrolein, alpha-n-propyl acrolen; dialdehydes, e.g., glutaraldehyde, succinaldehyde or their derivatives or their mixtures with additives capable of copolymerization (comonomers), e.g., alpha-substituted acroleins, beta-substituted acroleins, ethyl cyanoacrylates, methyl cyanoacrylates, butyl cyanoacrylates, hexyl eyanoacrylate, methyl methacrylates, vinyl alcohols, acrylic acids, methacrylic acids, acrylic acid chlorides, methacrylic acid chlorides, acrylonitrile, methacrylonitriles, acrylamides, substituted acrylamides, hydroxy methyl methacrylates, mesityl oxide, dimethylaminoethylmethacrylates 2-vinylpyridines and N-vinyl-2-pyrrolidinone.

Suitable coupling agents that may be employed in the synthesis of PEG copolymers include, but are not limited to: compounds containing amino groups (e.g., hydroxylamine, butylamine, allylamine, ethanolamine, trishydroxymethylaminomethane, 3-amino-1-propanesulfonic acid, 5-aminovaleric acid, 8-aminooetanoic acid, D-glucosamine hydrochloride, aminogalactose, aminosorbitol, aminomannitol, diethylaminoethylamine, anilines, sulfonyl acid amide, choline, N-methylglucamine, piperazine, 1,6-hexanediamine, urea, hydrazine, glycine, alanine, lysine, serine, valine, leucine, peptides, proteins, albumin, human serum albumin, polylysine, gelatin, polyglycolamines, aminopolyalcohols, dextran sulfates with amino groups, N-aminopolyethylene glycol (HO-PEG-NH₂), N,N′-diaminopolyethylene glycol (NH₂-PEG-NH₂), antibodies, immunoglobulins, etc.); compounds containing acid groups, e.g., carboxylic acids such as acetic acid, propionic acid, butyric acid, valeric acid, caproic acid, caprylic acid, capric acid, lauric acid, myristic acid, palmitic acid, stearic acid, oleic acid, linolic acid, linolenic acid, cyclohexane carboxylic acid, phenylacetic acid, benzoylacetic acid, chlorobenzoic acid, bromobenzoic acid, nitrobenzoic acid, ortho-phthalic acid, meta-phthalic acid, para-phthalic acid, salicylic acid, hydroxybenzoic acid, aminobenzoic acid, methoxybenzoic acid, PEG-linker-glutaminic acid, PEG-linker-DTPA, PEG-linker-EDTA, etc.; compounds containing hydroxy groups, i.e., alcohols such as methanol, ethanol, propanol, butanol, pentanol, hexanol, heptanol, octanol, decanol, dodecanol, tetradecanol, hexadecanol, octadecanol, isopropyl alcohol, isobutyl alcohol, isopentyl alcohol, cyclopentanol, cyclohexanol, crotyl alcohol, benzyl alcohol, phenyl alcohol, diphenyl methanol, triphenyl methanol, cinnamyl alcohol, ethylene glygol, 1,3-propanediol, glycerol, pentaerythritol and the like; polymerizable substances, such as alpha, beta-unsaturated aldehydes, e.g., acrolein, crotonaldehyde, propionaldehyde, etc.; alpha-substituted acrolein derivatives, e.g., alpha-methylacrolein, alpha-chloroacrolein, alpha-phenylacrolein, alpha-ethylacrolein, alpha-isopropylacrolein, alpha-n-butylacrolein, alpha-n-propylacrolein, etc.; and dialdehydes, e.g., glutaraldehyde, succinaldehyde or their derivatives or their mixtures with additives capable of copolymerization, such as alpha-substituted acroleins, beta-substituted acroleins, ethyl cyanoacrylates, methyl cyanoacrylates, butyl acrylates, hexyl cyanoacrylates, methylmethacrylates, vinyl alcohols, acrylic acids, methacrylic acids, acrylic acid chlorides, acrylonitrile, methacrylonitriles, acrylamides, substituted acrylamides, hydroxymethylmethacrylates, mesityl oxide, dimethylaminoethylmethacrylates 2-vinylpyridines, vinylpyrrolidinone, etc.

Particularly preferred coupling agents include hydroxylamine, trishydroxymethylaminomethane, 3-amino-1-propane sulfonic acid, D-glucosaminohydrochloride, aminomannitol, urea, human serum albumin, hydrazine, proteins, polyglycolamines, aminopolyalcohols (e.g., HO-PEG-NH₂ or NH₂-PEGNH₂), and compounds containing acid groups such as PEG-linker-asparaginic acid, PEG-linker-glutaminic acid, PEG-linker-DTPA and PEG-linker-EDTA, wherein the molecular weight of the PEG is less than about 100 kD, preferably less than about 40 kD.

The amount of coupling agent is typically present in the range of about 1 to about 95 wt % of the polyaldehyde in the PEG copolymer. The coupling agents can be condensed by their amino group or on the formyl groups located on the surface of nanoparticles synthesized from polymerized aldehydes and optional surfactants. Also, such formyl groups may also bind those monomers listed above that are polymerizable. However, the acids and alcohols named above are typically coupled on the nanoparticles only after previous conventional conversion of the aldehyde function.

The polymer may also contain hydrolyzable linkages to enable hydrolytic degradation within the body and thus facilitate drug release from the polymeric matrix. Suitable hydrolyzable linkages include any intramolecular bonds that can be cleaved by hydrolysis, typically in the presence of acid or base. Examples of hydrolyzable linkages include, but are not limited to, those disclosed in WO 99/22770 to Harris, such as carboxylate esters, phosphate esters, acetals, imines, ortho esters and amides.

Other suitable hydrolyzable linkages include, for example, enol ethers, diketene acetals, ketals, anhydrides and cyclic diketenes. Formation of such hydrolyzable linkages within the hydrophilic polymer is conducted using routine chemistry known to those skilled in the art of organic synthesis and/or described in the pertinent texts and literature. For example, carboxylate linkages may be synthesized by reaction of a carboxylic acid with an alcohol, phosphate ester linkages may be synthesized by reaction of a phosphate group with an alcohol, acetal linkages may be synthesized by reaction of an aldehyde and an alcohol, and the like. Thus, polyethylene glycol containing hydrolyzable linkages “X” might have the structure -PEG-X-PEG- or alternatively might be a matrix having the structure

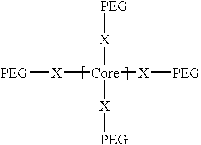

wherein the core is hydrophobic molecule such as pentaerythritol, may be synthesized by reaction of various -PEG-Y molecules with -Core-Z or PEG-Z molecules wherein Z and Y represent groups located at the terminus of individual PEG molecules and are capable of reacting with each other to form the hydrolyzable linkage X.

Accordingly, it will be appreciated that the rate of drug release from the polymeric matrix can be controlled by adjusting the degree of branching of the polymer, by incorporating different types of monomer units in the polymer structure, by functionalizing the polymer with different terminal species (which may or may not be charged), and/or by varying the density of hydrolyzable linkages present within the polymeric structure.

As noted above, depending on the particular polymer employed, the polymers may be relatively more hydrophilic or relatively more hydrophobic. Examples of suitable, relatively more hydrophilic polymers include, but are not limited to, polyethylene glycol, polypropylene glycol, branched polyethylene imine, polyvinyl pyrrolidone, polylactide, poly(lactide-co-glycolide), polysorbate, polyethylene oxide, poly(ethylene oxide-co-propylene oxide), poly(oxyethylated) glycerol, poly(oxyethylated) sorbitol, poly(oxyethylated glucose), polymethyloxazoline, polyethyloxazoline, polyhydroxyethyloxazoline, polyhydroxypropyloxazoline, polyvinyl alcohol, poly(hydroxyalkylcarboxylic acid), polyhydroxyethyl acrylic acid, polyhydroxypropyl methacrylic acid, polyhydroxyvalerate, polyhydroxybutyrate, polyoxazolidine, polyaspartamide, polysialic acid, and derivatives, mixtures and copolymers thereof.

Examples of suitable, relatively more hydrophobic polymers include linear polypropylene imine, polylactide, polyglycolide, polyethylene sulfide, polypropylene sulfide, polyethylenesulfonate, polypropylenesulfonate, polyethylene sulfone, polyethylenesulfonylethyleneimine, polycaprolactone, polypropylene oxide, polyvinylmethylether, polyhydroxyethyl acrylate, polyhydroxypropyl methacrylate, polyphosphazene and derivatives, mixtures and copolymers thereof.

Preferred among the foregoing polymers for use in the present compositions are polyethylene glycol (PEG), polypropylene glycol (PPG), and copolymers of PEG and PPG, or PEG and/or PPG containing some fraction of other monomer units (e.g., other alkylene oxide segments such as propylene oxide). Other preferred copolymers are branched copolymers containing PEG and caprolactone, PEG and lactide, and PEG and lactide-co-glycolide where the core is comprised of either the more hydrophilic or the more hydrophobic polymer. Another particularly preferred copolymer is a branched polymer of PEG and PPG, particularly wherein the PPG units comprise the innermost portion of the structure and the PEG units comprise the outer portions of the arms of the branched structure. Also preferred among the foregoing polymers are polysorbates, particularly polysorbate 80 (commercially available as TWEEN®80), sorbitan mono-9-octadecanoate poly(oxy-1,2-ethanediyl) derivatives.

As illustrated above, the branched PEG molecule may be modified to have a hydrophobic core. For example, if the central core is pentaerythritol, the innermost arms bound to the pentaerythritol may comprise a polymer more hydrophobic than PEG. Useful polymers to accomplish this include polypropylene glycol and polybutylene glycol. Useful monomers for constructing the inner, hydrophobic core structures of the arms include propylene oxide, butylene oxide, copolymers of the two, lactic acid and copolymers of lactic acid with glycolide (polylactide-co-glycolide and copolymers of the foregoing with polyethylene glycol). The preferred materials for constructing an inner hydrophobic core include polypropylene glycol and copolymers of propylene oxide with ethylene oxide. Useful polymers for constructing the outer, peripheral parts of the arms include polyethylene glycol, polycaprolactone, polylactide, poly[lactide-co-glycolide], polysialic acid and other hydrophilic polymers, with PEG most preferred. It is possible that a fraction of the monomers in the outer portion of a given arm of the carrier molecule may be replaced with PEG, but in this case, there will be substantially more of the hydrophilic monomer (e.g. ethylene oxide) than the hydrophobic monomer (e.g. propylene oxide).

The relative proportion of the hydrophobic polymer within the branched polymer may vary from about 10 to about 99 wt %, preferably from about 25 to about 95 wt %. When more hydrophobic polymer is used this may increase the drug loading capacity of the branched molecule for hydrophobic drugs. A most preferred ratio is about 10 wt % weight of hydrophobic polymer, e.g. polypropylene glycol, and 90 wt % weight ratio of hydrophilic polymer (e.g. PEG) in the outer arms.

The branched molecules comprising a hydrophobic core and peripheral hydrophilic arms are thought to have a number of advantages for drug delivery. The hydrophobic core may better stabilize hydrophobic drugs within the branched molecule and, as the drug is stabilized within the core, the free arms of the PEG may be better able to maintain a random state in which the PEG molecules move freely within solution. The outer, hydrophilic PEG layer may act as a steric barrier, inhibiting or decreasing the aggregation of individual branched molecules into particles. Additionally, in instances when targeting ligands are attached to the termini of the peripheral hydrophilic arms, targeting is facilitated by the unencumbered and exposed nature of the outer PEG arms. As will be discussed further on, a wide variety of targeting ligands can be covalently bound to the free ends of the PEG. The hydrophobic and hydrophilic components of the arms may be linked together by a variety of different. Such linkers include ethers, amides, esters, carbamates, thioesters, disulfide bonds. In general, the linker employed is used to attain the desired drug delivery properties of the pharmaceutical formulation. Metabolizable bonds can be selected to improve excretion of the carrier molecule as well as to improve drug release.

The branched molecules comprising a hydrophilic core and peripheral hydrophobic arms are also thought to have a number of advantages for drug delivery. The hydrophilic core may better solubilize hydrophobic drugs by forming a spatially stabilized matrix in which the hydrophobic moieties serve to sequester the drug and the hydrophilic moieties interact with the aqueous solution. Additionally, in instances when targeting ligands are attached to the termini of the peripheral hydrophobic arms, targeting is facilitated by the unencumbered and exposed nature of the outer polymer arms. As will be discussed further on, a wide variety of targeting ligands can be covalently bound to the free ends of the hydrophobic polymers. The hydrophilic and hydrophobic components of the arms may be linked together by a variety of different linkers. Such linkers include ethers, amides, esters, carbamates, thioesters, and disulfide bonds. In general, the linker employed is used to attain the desired drug delivery properties of the pharmaceutical formulation. Metabolizable bonds can be selected to improve excretion of the carrier molecule as well as to improve drug release.

In one embodiment of the present invention, the branched polymer comprises a block copolymer. The block copolymer may be a mixture of hydrophobic and hydrophilic blocks, tetronic, but preferentially with hydrolyzable bonds. The block copolymer may arise from a central core of, for example, a sugar molecule, a polysaccharide or a frame polymer. In a preferred form, the block copolymer preferably includes a central core from which radiate about 3 to 12 arms, with from about 4 to 8 arms preferred.

In one embodiment, each arm may comprise a block copolymer with an inner, more hydrophobic block and an outer, more hydrophilic block. In preferred embodiments, the inner block may comprise polypropylene oxide (PPO), polylactide (PLA), polylactide-coglycolide (PLGA) or b-polycaprolactone, and the outer block comprises polyethylene glycol, PEG-PPO, PEG-PLA, PEG-PLGA, and PEG-b-polycaprolactone, respectively. Also in preferred embodiments, the targeting ligands may be attached to the outermost portion of the arms. In another embodiment, each arm may comprise a block copolymer with an inner, more hydrophilic block and an outer, more hydrophobic block, also referred to as reverse block copolymers.

In certain embodiments, the polymer may have a multivalent core structure from which extend arms comprising linear or branched polymers. The cores may preferably be polyhydroxylated monomers such as sugars, sugar alcohols, polyaliphatic alcohols and the like. Preferred among such core structures are triethanolamine, which contains three hydroxyl moieties; and neopentanol and polyerythritol, which contain four hydroxy moieties that may be derivatized to afford the various arms or branches. Sugar alcohols such as glycerol, mannitol and sorbitol may also be similarly derivatized.

When a polymer is used as the stabilizing agent in the formulation of the invention, the amount of polymer, e.g. poloxamer or poloxamine, may range from about 0.1 to about 99 wt % of the formulation. More preferably the polymer will range from about 10 to about 90 wt % and still more preferably from about 10 to about 50 wt % of the formulation. Preferred ratios of polymer to drug range from 5:1 to approximately 1:100, with ranges of 1:3 to 1:7 being preferred.

The molecular weight of the polymer employed in the present compositions may vary depending, for example, upon the particular polymer selected, the particular therapeutic agent selected, the desired rate of release, and the like. Broadly speaking, the molecular weight of the polymer may range from about 1,000 to about 1,000,000 (and all combinations and subcombinations of ranges and specific molecular weights therein). More preferably, the polymer may have a molecular weight of from about 4,000 to about 50,000, with molecular weights of from about 10,000 to about 20,000 being even more preferred, and a molecular weight of about 12,000 being particularly preferred. Examples of lower molecular weight polymers include polymers such as TWEEN®80 (about 1,200 daltons) or small branched PEGs on the order of from about 1000 to about 2000 daltons.

In an alternate embodiment of the present invention, the polymers employed in the compositions described herein may be polypeptides, i.e., the polymers may comprise repeating units of amino acids. Certain advantages may be achieved in embodiments employing polypeptides in the compositions of the present invention, particularly in embodiments in which hydrophobic domain(s) of the matrices comprise polypeptides. In this connection, peptides may be biodegradable, for example, via the action of enzymes in the body, such as esterases and amidases. Thus, matrices which include polypeptides may exhibit improved metabolism and/or reduced toxicity in the body. In addition, different amino acids or groups of amino acids may be selected, for example, to optimize the interaction of the therapeutic agents with the polymeric matrix. For example, amino acids may be selected such that the polypeptide may form a tertiary structure that facilitates wrapping, folding and/or envelopment of the polymer around the drug. Polyleucine, for example, may form an α-helical structure, that may wrap around a hydrophobic active agent to basically form a tube or tubule around the active agent. The polypeptides employed in the present compositions may be prepared by modern synthetic methods, such as solid phase synthesis and recombinant techniques.

In the case of hydrophobic active agents, polypeptides comprising hydrophobic amino acids may generally be employed, for example, to form a block within the block copolymer, which may preferably comprise both hydrophobic and hydrophilic domains. The polypeptides may be derived from natural, L and D amino acids, as well as unnatural and modified amino acids. In addition, the polypeptides may be fluorinated, i.e., the polypeptides may be substituted with fluorine atoms or fluorinated groups to provide amino acids and polypeptides having a higher degree of hydrophobicity. For example, naturally occurring hydrophobic amino acids, including leucine, isoleucine, valine, proline, alanine, tyrosine and tryptophan, may be used, for example, to provide a homopolymer or a heteropolymer comprising a fragment of hydrophobic amino acids in a polypeptide. The hydrophobic polypeptide may then be covalently attached to a different polymer, for example, a hydrophilic polymer, including the hydrophilic polymers described herein, which in turn may preferably be attached to a targeting ligand, as discussed in detail below.

The length of the polypeptide as well as the particular amino acids employed may be selected, for example, to optimize the interaction between the polypeptide and the active agent including, for example, the extent and the manner in which the polypeptide may envelop, fold or wrap around the active agent. For example, in the case of polyleucine, other amino acids, such as, for example, glycine or proline, may be incorporated into the polypeptide to modify the way the polypeptide bends which may permit increased and more efficient wrapping of the polypeptide around the active agent. Similarly, domains of amino acids may be selected and incorporated in the polypeptide which may improve the chemical interaction or association with the active agent. For example, the drug irinotecan is a lipophilic cation, and the drug camptothecin is hydrophobic although the pyridine residue may be attached to the 10-hydroxy position of camptothecin to provide a pro-drug. The pyridine moiety may also carry a positive charge at physiological pH from the quaternary amine. Incorporating one or more anionic amino acids, for example, glutamate, into the polyleucine polypeptide, may serve to increase the interaction of the predominantly polyleucine polypeptide with camptothecin. In general, for active agents such as irinotecan, which are lipophilic cations, incorporating an anionic segment into the polypeptide may increase the interaction. Conversely, for active agents that are lipophilic anions, one or more cationic amino acids, for example, lysine, arginine or histidine, may be incorporated into the polypeptide. Without intending to be bound by any theory or theories of operation, it is contemplated that the polypeptide may serve as a hydrophobic block which facilitates hydrogen bonding with a active agent containing a charged domain, thereby enabling the formation of a complex, or some other interaction, for example, ion pairing of the polypeptide with the polar, charged portion of the active agent.

While a hydrophobic polypeptide may form a complex or provide other interaction with a given active agent, this is generally insufficient to solubilize the active agent, unless a segment of hydrophilic amino acids is also incorporated into the polypeptide or the polypeptide is otherwise modified, for example, derivatized, to incorporate hydrophilic groups. Solubilization of the hydrophobic active agent/polypeptide matrix may be accomplished, for example, by creating within the polypeptide, not only a block of hydrophobic amino acids, but also a block of hydrophilic or charged amino acids proximate the hydrophobic block. Preferably, however, the hydrophobic segment of amino acids may be covalently bound to another polymer, preferably a hydrophilic polymer, such as polyethyleneglycol (PEG). For example, a decapeptide of polyleucine may be attached to a hydrophilic polymer, such as PEG, for example, via the free amino end of the polyleucine peptide and the free carboxyl end of α-amino, γ-carboxy PEG. The free end of the PEG, via its amino group, may then be used to attach a targeting ligand, for example, a peptide via its terminal carboxyl group. In such embodiments, the hydrophilic polymer, for example, PEG, may vary in length such that it's molecular weight may range, for example, from about 400 to about 100,000 daltons, with molecular weights of from about 1,000 to about 40,000 being preferred. More preferably, the molecular weight of the hydrophilic polymer in the context of the present embodiment, is about 3,500 daltons. Generally speaking, a hydrophilic polymer, such as PEG, having a higher molecular weight, may afford a longer circulation lifetime, but may decrease the affinity of the targeted matrix as the molecular weight increases. Therefore, the molecular weight of the hydrophilic polymer may be is selected for the particular application. It should be noted that, in embodiments involving linear polypeptides, the polymer may be attached to one or both ends of the polypeptide, i.e., to both α-amino and γ-carboxy end groups. Similarly, in the case of attachment of a polymer to both termini of the polypeptide, then the targeting ligand(s) may be attached to one or both termini of the polypeptide-polymer conjugate.

The length of the segment of amino acids in the polypeptide may vary depending, for example, upon the intended application, and the chemistry of the active agent to be delivered, the size of the active agent to be delivered, and the like. In general, at least one hydrophobic amino acid may preferably be incorporated into the polypeptide, but generally the number of amino acids incorporated into the polypeptide may range from about 3 to 100 amino acids (and all combinations and subcombinations of ranges and specific numbers of amino acids therein). Preferably, the polypeptide comprises from about 5 to 20 amino acids, with about 10 amino acids being more preferred.

As with the other polymers, including hydrophilic polymers discussed above, the polypeptides may be linear or branched. To create a branched block polypeptide, amino acids with side chains may be used, for example, to first create a backbone. For example, one may start with a backbone of branching amino acids utilizing, for example, the epsilon amino moiety of polylysine or the side chain carboxyl moiety of polyglutamic acid. The backbone may comprise a homopolymer of amino acids or a copolymer of amino acids. Copolymers may be advantageous, for example, in that one or more amino acids can be used as “spacers” to increase the distance between side chains, and thereby minimize steric hindrance or to otherwise optimize properties of the backbone. For example, the backbone may comprise an alternating sequence of lysine with glycine or another amino acid so as to increase the spacing between the side chain bearing amino acids. Preferably, however, when a backbone of branched amino acids is employed, the polymer is in the form of a homopolymer, for example, polylysine or polyglutamate. When a backbone is prepared from the branched amino acids, using peptide chemistry, hydrophobic blocks in the form of pendant peptides may then be attached to the activated side chains of the backbone. In so doing, a branching structure may be created which comprises a plurality of hydrophobic domains. Hydrophilic polymers, such as PEG, may then in turn be attached to the free ends of the pendant chains of hydrophobic amino acids to create a branched block polymer comprised of amino acids and PEG. When such a structure is created from a backbone and multiple chains, then the structure preferably has from about 3 to 100 arms, more preferably from about 4 to 20 arms, and still more preferably from about 4 to 8 arms.

2. Lipid Stabilizing Agents

Useful lipids include phospholipids and lecithins, where the phospholipid can be a natural phospholipid, a chemically or enzymatically modified phospholipid, or a synthetic phospholipid. Examples of suitable lipids include, but are not limited to, phosphatidylglycerol, phosphatidic acid, phosphatidylserine, phosphatidylinositol, cerebrosides, gangliosides, sphingosines, cardiolipin, and sulfatides.

Other suitable phospholipids include diacyl phospholipids such as diacyl derivatives of phophatidylcholine (diacyl phosphatidylcholines), phosphatidylethanolamine (diacyl phosphatidylethanolamines), phosphatidylserine (diacyl phosphatidylserines), phosphatidylglycerol (phosphorylated diacylglycerides), phosphatidylinositol (diacyl phosphatidylinositols and phosphatidic acid (diacyl phosphatidic acids), and combinations thereof. The fatty acyl chain may be from 10 to 22 carbons in length and may be saturated, monounsaturated, or polyunsaturated. The fatty acid at the 1 and 2 positions may be mixed or the same in the acylglyceryl moieties. Preferred saturated fatty acyl moieties include lauryl, myristyl, palmityl, stearyl, or higher chain derivatives; preferred unsaturated acyl moieties include oleyl chains. A given phospholipid may contain two identical chains, as in DOPE (dioleylphosphatidylethanolamine), or mixed chains as in POPE (1-palmitoyl-2-oleylphosphatidylethanolamine).

Exemplary diacyl phosphatidylcholines include, by way of example, palmitoylolcoyl phosphatidylcholine (POPC), dioleoyl phosphatidylcholine (DOPC), dilauroyl phosphatidylcholine (DLPC), dimyristoyl phosphatidylcholine (DMPC), dipalmitoyl phosphatidylcholine (DPPC), distearoyl phosphatidylcholine (DSPC), and combinations thereof. Exemplary diacyl phosphatidylethanolamines include, by way of example, dipalmitoyl phosphatidylethanolamine (DPPE), 1-palmitoyl-2-olcoylphosphatidylethanolamine (POPE), dioleylphosphatidylethanolamine (DOPE), and combinations thereof. Exemplary phosphorylated diacylglycerides include, for example, dioleoyl phosphatidylglycerol (DOPG), palmitoyloleyl phosphatidylglycerol (POPG), and combinations thereof. POPG is a particularly preferred lipid.

When a lipid is employed as the stabilizing agent in the formulation, the amount of lipid may range from about 0.1 to about 99 wt % of the formulation. More preferably the lipid will range from about 1 to about 90 wt % and still more preferably from about 2 to about 50 wt % of the formulation. Preferred ratios of lipid to drug range from 5:1 to 2.5:1 to approximately 1:1, with ranges of 2:1 to 1:1 being preferred. In one embodiment, the lipid to drug weight ratio is less than 5:1, more preferably less than 3:1, and most preferably less than 1:1.

3. Polymer-Lipid Conjugate Stabilizing Agents

As indicated above, the polymers employed in the present compositions may be linked or conjugated to a lipid, preferably a phospholipid, to provide a polymer-lipid conjugate, as in the case, for example, of PEG-phospholipid conjugates (also referred to as “PEGylated” phospholipids).

The polyethylene glycol in the PEGylated phospholipids may be branched, star or linear, and may be derivatized with amino, carboxyl, acyl, or sulfonyl ends. Conjugates of linear PEG and phospholipids, if used, will generally although not necessarily employ PEG have a molecular weight in the range of approximately 100-50,000 daltons, preferably in the range of approximately 350-40,000 daltons, more preferably 350-7000 daltons, and most preferably from 750-5,000 daltons. It will be appreciated by those skilled in the art that the aforementioned molecular weight ranges correspond to a polymer containing approximately 2-1000 ethylene oxide units, preferably about 8 to 1000 ethylene oxide units. The phospholipid moiety that is conjugated to the PEG may be anionic, neutral or cationic, of naturally occurring or synthetic origin, and normally comprises a diacyl phosphatidylcholine, a diacyl phosphatidylethanolamine, a diacyl phosphatidylserine, a diacyl phosphatidylinositol, a diacyl phosphatidylglycerol, or a diacyl phosphatidic acid, wherein each acyl moiety can be saturated or unsaturated and will generally be in the range of about 10 to 22 carbon atoms in length.

Exemplary PEGylated” phospholipids include, by way of example, diacyl lipid-PEG conjugates such as DPPE-PEG, DOPE-PEG, POPE-PEG, where the PEG length can vary so as to provide for a PEG molecular weight of 2 kDa, 5 kDa, 10 kDa, and greater. In addition, PEG can be conjugated to a fatty acid, for example, such as a myrj compound, e.g., myrj 52.

Preferred compounds are polymer-conjugated diacyl phosphatidyl-ethanolamines having the structure of formula (I)

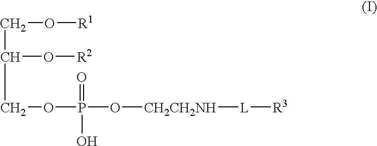

wherein R¹ and R² are the acyl groups, R³ represents the hydrophilic polymer, e.g., a polyalkylene oxide moiety such as poly(ethylene oxide) (i.e., polyethylene glycol), poly(propylene oxide), poly(ethylene oxide-co-propylene oxide) or the like (for linear PEG, R³ is —O—(CH₂CH₂O)ₙ—H), and L is an organic linking moiety such as a carbamate, an ester, an amide, an amine, an imine, or a diketone having the structure of formula (II)

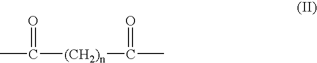

wherein n is 1, 2, 3 or 4. Preferred unsaturated acyl moieties are esters formed from oleic and linoleic acids, and preferred saturated acyl moieties are palmitate, myristate and stearate. Particularly preferred phospholipids for conjugation to linear, branched or star PEG herein are dipalmitoyl phosphatidylethanolamine (DPPE) and 1-palmitoyl-2-oleyl phosphatidylethanolamine (POPE).

The conjugates may be synthesized using art-known methods such as described, for example, in U.S. Pat. No. 4,534,899 to Sears. That is, synthesis of a PEG-phospholipid conjugate or a conjugate of a phospholipid and an alternative hydrophilic polymer may be carried out by activating the polymer to prepare an activated derivative thereof, having a functional group suitable for reaction with an alcohol, a phosphate group, a carboxylic acid, an amino group or the like. For example, a polyalkylene oxide such as PEG may be activated by the addition of a cyclic polyacid, particularly an anhydride such as succinic or glutaric anhydride (ultimately resulting in the linker of formula (II) wherein n is 2 or 3, respectively). The activated polymer may then be covalently coupled to the selected phosphatidylalkanolamine, such as phosphatidylethanolamine, to give the desired conjugate.

B. The Active Agent

The drug in the formulation, as noted above, is any active agent whose systemic bioavailability can be enhanced by increasing the solubility of the agent in water.

Generally speaking, the active agent may have a limited water solubility. The term “limited water solubility,” as used herein, means that the active agent may be sparingly soluble in aqueous systems, and may exhibit a degree of solubility in systems having increased hydrophobicity, such as polymers, including the polymers described herein. In one embodiment, the ratio of the solubility of the drug in the polymer to the solubility of the drug in water is greater than about 1:1. More preferably, the ratio of the solubility of the drug in the polymer to the solubility of the drug in water is at least about 10:1.

Any number of drugs may be incorporated into the formulations of the invention, i.e., any compounds that fit the aforementioned solubility criteria and induce a desired systemic effect. Such substances include the broad classes of compounds normally administered systemically. In general, this includes: analgesic agents; antiarthritic agents; respiratory drugs, including antiasthmatic agents and drugs for preventing reactive airway disease; antibiotics; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; antiinflammatory agents; antimetabolic agents; antimicrobial agents; antimigraine preparations; antinauseants; antiparkinsonism drugs; antipruritics; antipsychotics; antipyretics; antispasmodics; antiviral agents; anxiolytics; attention deficit disorder (ADD) and attention deficit hyperactivity disorder (ADHD) drugs; cardiovascular preparations including cardioprotective agents; central nervous system stimulants; cough and cold preparations, including decongestants; diuretics; genetic materials; gonadotropin releasing hormone (GnRH) inhibitors; herbal remedies; hormonolytics; hypnotics; immunosuppressive agents; leukotriene inhibitors; mitotic inhibitors; muscle relaxants; parasympatholytics; peptide drugs; psychostimulants; sedatives; steroids; sympathomimetics; tranquilizers; vasodilators, including peripheral vascular dilators; and vitamins.

It will be appreciated that the invention is particularly useful for delivering active agents for which chronic administration may be required, as the present formulations provide for sustained release. The invention is thus advantageous insofar as patient compliance with regard to forgotten or mistimed dosages is substantially improved. Any agent that is typically incorporated into a capsule, tablet, troche, liquid, suspension or emulsion, wherein administration is on a regular (i.e., daily, more than once daily, every other day, or any other regular schedule) can be advantageously delivered using the formulations of the invention.

Examples of drugs for which a sustained release formulation is particularly desirable include, but are not limited to, the following:

analgesic agents: hydrocodone, hydromorphone, levorphanol, oxycodone, oxymorphone, codeine, morphine, alfentanil, fentanyl, meperidine and sufentanil, diphenylheptanes such as levomethadyl, methadone and propoxyphene, and anilidopiperidines such as remifentanil;

antiandrogens: bicalutamide, flutamide, hydroxyflutamide, zanoterine and nilutamide;

anxiolytic agents and tranquilizers: diazepam, alprazolam, chlordiazepoxide, clonazepam, halazepam, lorazepam, oxazepam and clorazepate;

antiarthritic agents: hydroxychloroquine, gold-based compounds such as auranofin, aurothioglucose and gold thiomalate, and COX-2 inhibitors such as celecoxib and rofecoxib;

antibiotics (including antineoplastic antibiotics): vancomycin, bleomycin, pentostatin, mitoxantrone, mitomycin, dactinomycin, plicamycin and amikacin;

anticancer agents, including antineoplastic agents: paclitaxel, docetaxel, camptothecin and its analogues and derivatives (e.g., derivatives at the 9 ring position, including 9-nitro-camptothecin and 9-amino-camptothecin; modified 10-position compounds, including 10-hydroxycamptothecin and aminated, aminoalkyl, alkylated, and alkoxylated derivatives of the same; modified 11-position derivatives, including 11-hydroxycamptothecin and aminated, aminoalkyl, alkylated, and alkoxylated derivatives of the same; modified 12-position derivatives, including 12-hydroxycamptothecin and aminated, aminoalkyl, alkylated, and alkoxylated derivatives of the same; 7-position derivatives, including amino, nitro, alkyl, alkylamino, and alkoxy derivatives of the same; 20 (S) derivatives such as 20-O-β-glucopyranosyl camptothecin, including alkylesters and amides of the 20-(OH) group; Camptotheca alkaloids including, but not limited to, homocamptothecin, diflomotecan, exatecan, topotecan, irinotecan, and carzelesin; and metabolites such as 7-ethyl-10-hydroxycamptothecin, designated SN-38, a metabolite of irinotecan), taxanes (baccatins, cephalomannine and their derivatives), carboplatin, cisplatin, interferon-α_{2A}, interferon-α_{2B}, interferon-α_{N3} and other agents of the interferon family, levamisole, altretamine, cladribine, bovine-calmette-guerin (BCG), aldesleukin, tretinoin, procarbazine, dacarbazine, gemcitabine, mitotane, asparaginase, porfimer, mesna, amifostine, mitotic inhibitors including podophyllotoxin derivatives such as teniposide and etoposide and vinca alkaloids such as vinorelbine, vincristine and vinblastine; If you have access to a database of updated, new anti-cancer drugs it would be good to expand this.

antidepressant drugs: selective serotonin reuptake inhibitors such as sertraline, paroxetine, fluoxetine, fluvoxamine, citalopram, venlafaxine and nefazodone; tricyclic anti-depressants such as amitriptyline, doxepin, nortriptyline, imipramine, trimipramine, amoxapine, desipramine, protriptyline, clomipramine, mirtazapine and maprotiline; other anti-depressants such as trazodone, buspirone and bupropion;

antiestrogens: tamoxifen, clomiphene and raloxifene;

antifungals: amphotericin B, imidazoles, triazoles, and griesofulvin;

antihyperlipidemic agents: HMG-CoA reductase inhibitors such as atorastatin, simvastatin, pravastatin, lovastatin and cerivastatin sodium, and other lipid-lowering agents such as clofibrate, fenofibrate, gemfibrozil and tacrine;

antimetabolic agents: methotrexate, fluorouracil, floxuridine, cytarabine, mercaptopurine and fludarabine phosphate;

antimigraine preparations: zolmitriptan, naratriptan, sumatriptan, rizatriptan, methysergide, ergot alkaloids and isometheptene;

antipsychotic agents: chlorpromazine, prochlorperazine, trifluoperazine, promethazine, promazine, thioridazine, mesoridazine, perphenazine, acetophenazine, clozapine, fluphenazine, chlorprothixene, thiothixene, haloperidol, droperidol, molindone, loxapine, risperidone, pimozide and domepezil;

aromatase inhibitors: anastrozole and letrozole;

attention deficit disorder and attention deficit hyperactivity disorder drugs: methylphenidate and pemoline;

cardiovascular preparations: angiotensin converting enzyme (ACE) inhibitors; diuretics; pre- and afterload reducers; cardiac glycosides such as digoxin and digitoxin; inotropes such as amrinone and milrinone; calcium channel blockers such as verapamil, nifedipine, nicardipene, felodipine, isradipine, nimodipine, bepridil, amlodipine and diltiazem; beta-blockers such as pindolol, propafenone, propranolol, esmolol, sotalol and acebutolol; antiarrhythmics such as moricizine, ibutilide, procainamide, quinidine, disopyramide, lidocaine, phenyloin, tocainide, mexiletine, flecainide, encainide, bretylium and amiodarone; cardioprotective agents such as dexrazoxane and leucovorin;

GnRH inhibitors and other hormonolytics and hormones: leuprolide, goserelin, chlorotrianisene, dinestrol and diethylstilbestrol;

herbal remedies: melatonin;

immunosuppressive agents: 6-thioguanine, 6-aza-guanine, azathiopurine, cyclosporin and methotrexate;

lipid-soluble vitamins: tocopherols and retinols;

leukotriene inhibitors: zafirlukast, zileuton and montelukast sodium;

nonsteroidal anti-inflammatory drugs (NSAIDs): diclofenac, flurbiprofen, ibuprofen, ketoprofen, piroxicam, naproxen, indomethacin, sulindac, tolmetin, meclofenamate, mefenamic acid, etodolac, ketorolac and bromfenac;

peptide drugs: leuprolide, somatostatin, oxytocin, calcitonin and insulin;

peripheral vascular dilator: cyclandelate, isoxsuprine and papaverine;

respiratory drugs: such as theophylline, oxytriphylline, aminophylline and other xanthine derivatives;

steroids: progestogens such as flurogestone acetate, hydroxyprogesterone, hydroxyprogesterone acetate, hydroxyprogesterone caproate, medroxyprogesterone acetate, megestrol, norethindrone, norethindrone acetate, norethisterone, norethynodrel, desogestrel, 3-keto desogestrel, gestadene and levonorgestrel; estrogens such as estradiol and its esters (e.g., estradiol benzoate, valerate, cyprionate, decanoate and acetate), ethynyl estradiol, estriol, estrone, mestranol and polyestradiol phosphate; corticosteroids such as betamethasone, betamethasone acetate, cortisone, hydrocortisone, hydrocortisone acetate, corticosterone, fluocinolone acetonide, flunisolide, fluticasone, prednisolone, prednisone and triamcinolone; androgens and anabolic agents such as aldosterone, androsterone, testosterone and methyl testosterone; and

topoimerase inhibitors: camptothecin, anthraquinones, anthracyclines, teniposide, etoposide, topotecan and irinotecan.

Genetic material may also be delivered using the present formulation, e.g., a nucleic acid, RNA, DNA, recombinant RNA, recombinant DNA, antisense RNA, antisense DNA, hammerhead RNA, a ribozyme, a hammerhead ribozyme, an antigene nucleic acid, a ribooligonucleotide, a deoxyribonucleotide, an antisense ribooligonucleotide, and an antisense deoxyribooligonucleotide. Representative genes include vascular endothelial growth factor, fibroblast growth factor, BCl-2, cystic fibrosis transmembrane regulator, nerve growth factor, human growth factor, erythropoeitin, tumor necrosis factor, interleukin-2 and histocompatibility genes such as HLA-B7.

The foregoing list is merely illustrative and is not intended to be limiting. A wide variety of drugs and drug types can be effectively administered using the present formulations, although the invention is most advantageous with hydrophobic drugs.

It may be desirable to include one or more P-glycoprotein inhibitors in the formulation along with the active agent to be administered. It has been established that intestinal absorption of certain drugs, of which paclitaxel is exemplary, is controlled by P-glycoprotein (P-gp). With such drugs, then, the present formulations preferably include a P-gp inhibitor for oral administration in order to increase intestinal absorption and thus oral bioavailability. A particularly preferred P-gp inhibitor is cyclosporin A, although other P-gp inhibitors may also be used. When a P-gp inhibitor is included in the formulation, the weight ratio of drug to P-gp inhibitor (e.g., the ratio of paclitaxel to cyclosporin A) will generally be in the range of about 1:5 to 5:1, preferably in the range of about 1:2 to 2:1, more preferably in the range of about 1:1.5 to 1.5:1, and optimally about 1:1. With paclitaxel, it may also be desirable to co-administer a folate (i.e., a salt or ester of folic acid), which has been found to increase paclitaxel absorption.

The amount of drug in the formulation should be such that the weight ratio of drug to all other components of the formulation is in the range of about 1:1 to 1:50, preferably in the range of about 1:1 to 1:20, more preferably in the range of about 1:2 to 1:10, and optimally about 1:5.

C. Targeting Ligands

As noted above, the compositions of the present invention may also contain comprise one or more targeting ligands. A wide variety of targeting ligands may be employed in the present compositions depending, for example, on the particular tissue, cell or receptor to be targeted, the particular active agent and/or polymer employed, and the like. Generally speaking, materials which may be employed as targeting ligands include, for example, proteins such as antibodies, peptides, polypeptides, cytokines, growth factors and fragments thereof, vitamins and vitamin analogues such as folate, vitamin-B12, vitamin B6, niacin, nicotinamide, vitamin A and retinoid derivatives, ferritin and vitamin D, sugar molecules and polysaccharides, glycopeptides and glycoproteins, steroids, steroid analogs, hormones, cofactors, bioactive agents, and genetic material, including nucleosides, nucleotides and polynucleotides, drug molecules such as cyclosporin-A, prostaglandin and prostacyclin, and antagonists of the GPIIBIIIA receptor of platelets. Other suitable targeting ligands and methods of synthesizing and attaching such ligands are also described in WO 01/49268 to Unger et al.

In one embodiment, the targeting ligands employed in the present compositions may be covalently associated with the polymer. When multiple targeting ligands are attached to the polymer, the targeting ligands may comprise the same or different ligands. The number of targeting ligands attached to each polymer may vary, depending, for example, on the particular tissue, cells or receptors to be targeted, the targeting ligand and/or polymer selected, and the like. Generally speaking, the number of targeting ligands employed may range from less than about one targeting ligand per polymer molecule to a plurality of targeting ligands per polymer molecule including, for example, up to about several hundred targeting ligands per polymer molecule (and all combinations and subcombinations of ranges and specific numbers of targeting ligands therein). For example, in embodiments in which the matrices comprise nanoparticles, there may be as few as about 1 targeting ligand molecule per every 10 polymer molecules. Generally, the targeting ligands may be covalently attached to any portion of the polymer which may be available to form a covalent bond with a portion of the targeting ligand. For example, the targeting ligands may be covalently attached to the free ends of the polymer molecules, the free ends of the arms of branched polymer molecules, and/or the free ends of arms of star polymer molecules. In the case of branched polymers, the number of targeting ligands attached to the free ends of the branched polymer molecules may vary from less than about one to up to about one hundred targeting ligands per polymer molecule. Preferably, the number of targeting ligands may be about the same as the number of free arms in the branched polymer molecule. For example, in the compositions of the present invention, a branched PEG molecule containing 4 arms may also preferably contain 4 covalently associated targeting ligands, preferably to provide one targeting molecule per arm of PEG. As the branching of the polymer employed increases, the number of targeting ligands associated with the polymer may increase also. Although not preferred, the targeting ligands may also be bound to the backbone portion of the polymer molecules, rather than the free ends.

In certain embodiments, the targeting ligands employed in the compositions of the present invention may be peptides ranging from about 4 to 100 amino acids in length (and all combinations and subcombinations of ranges and specific numbers of amino acids therein). More preferably, the targeting ligands may comprise peptides ranging from about 4 to 20 amino acids in length, with from about 5 to 10 amino acids being even more preferred. Still more preferred are peptides containing about 6 or 7 amino acids, i.e., hexapeptides and heptapeptides.

Generally speaking, peptides that are particularly useful as targeting ligands include natural, modified natural, or synthetic peptides that incorporate additional modes of resistance to degradation by vascularly circulating esterases, amidases, or peptidases. The peptides may comprise D and L amino acids and mixtures of D and L amino acids, and may be comprised of all natural amino acids, all synthetic amino acids, and mixtures of natural and synthetic amino acids. The peptides may be synthesized on resins using solid phase synthetic chemistry techniques as are well known in the art, using solution phase chemistry or via recombinant techniques in which organisms such as yeast or bacteria are used to produce the peptide. In addition, peptides may be prepared by a variety of different combinatorial chemistry techniques as are now known in the art. One very useful method of stabilizing a peptide moiety incorporates the use of cyclization techniques. For example, end-to-end cyclization, whereby the carboxy terminus is covalently linked to the amine terminus via an amide bond, may be useful to inhibit peptide degradation and increase circulating half-life. Side chain-to-side chain cyclization may also be particularly useful in inducing stability. In addition, an end-to-side chain cyclization may be a useful modification as well. The substitution of an L-amino acid for a D-amino acid in a strategic region of the peptide may also provide resistance to biological degradation.

The targeting ligands may be incorporated in the present compositions in a variety of ways which would be apparent to the skilled artisan, once armed with the teachings of the present application. In preferred embodiments, the targeting ligands may be associated with other components of the present compositions, preferably the polymer, covalently. Peptides may be attached to the polymer molecules via their C-terminal or N-terminal groups or via side chains. Solid phase chemistry may be used to attach the peptides to the polymers, for example forming reactions on peptides preformed on a solid matrix, e.g. a resin. Alternatively, solution phase chemistry may be used to attach the peptides to the polymer molecules. Exemplary peptides and methods of linking them to the stabilizing agent are described in U.S. Patent Publication 2002/0041898 to Unger et al.

Antibodies may be used as whole antibodies or as antibody fragments, e.g., Fab or Fab′2, either of natural or recombinant origin. The antibodies of natural origin may be of animal or human origin, or may be chimeric (mouse/human). Human recombinant or chimeric antibodies are preferred and fragments are preferred to whole antibodies. Immunoglobulins typically comprise a flexible “hinge” region. See, e.g., “Concise Encyclopedia of Biochemistry,” Second Edition, Walter de Gruyter & Co., pp. 282-283 (1988). Antibodies may be linked to the termini of the outer hydrophilic arms using the thiols of this “hinge” region. This is a preferred region for coupling antibodies, as the potential binding site may be remote from the antigen-recognition site. Generally speaking, it may be difficult to utilize the thiols of the hinge group unless they are adequately prepared. As described in Shahinian et al. (1995) Biochimica et Biophysica Acta, 1239:157-167, it may be desirable to reduce the thiol groups so that they are available for coupling, e.g., to maleimide derivatized linking groups. Examples of reducing agents that may be used include ethanedithiol, mercaptoethanol, mercaptoethylamine or the more commonly used dithiothreitol, commonly referred to as Cleland's reagent. However, care should be exercised when utilizing certain reducing agents, such as dithiothreitol, as overreduction may compromise the activity or binding capacity of the targeting ligand.

Antibody fragments, such as F(ab′)₂, may be prepared by incubating the antibodies with pepsin (60 μg/ml) in 0.1 M sodium acetate (pH 4.2) for 4 h at 37° C. Digestion may be terminated by the addition of 2 M Tris (pH 8.8) to a final concentration of 80 mM. The fragments may then be obtained by centrifugation. The supernatant may be dialyzed at 4° C. against 150 mM NaCl, 20 mM phosphate at pH 7.0. Undigested IgG may be removed by chromatographic methods. The antibody fragments may then be extensively degassing the solutions and purging with nitrogen prior to use. The F(ab′)₂ fragments may be provided at a concentration of 5 mg/ml and reduced under argon in 30 mM cysteine. Alternatively, cysteamine maybe employed; 100 mM Tris, pH 7.6 maybe used as a buffer for 15 min at 37° C. The solutions may then be diluted 2-fold with an equal volume of the appropriate experimental buffer and spun through a 0.4 ml spin column of Bio-Gel P-6DG. The resulting antibody fragments may be more efficient in their coupling to the outer arms.

The same procedure may also be employed with other macromolecules containing cysteine residues for coupling to the termini of the PEG arms. Also, peptides may be utilized, especially if they contain a cysteine residue. If the peptides have not been made fresh and there is a possibility of oxidation of cysteine residues within the peptide structure, it may be necessary to regenerate the thiol group using the approach outlined above.

The attached targeting ligands may be directed toward lymphocytes that may be T-cells or B-cells, with T-cells being the preferred target. To select a class of targeted lymphocytes, a targeting ligand having specific affinity for that class may be preferably employed. For example, an anti CD-4 antibody may be used for selecting the class of T-cells harboring CD-4 receptors, an anti CD-8 antibody may be used for selecting the class of T-cells harboring CD-8 receptors, an anti CD-34 antibody may be used for selecting the class of T-cells harboring CD-34 receptors, and so on. A lower molecular weight ligand may preferably be employed, e.g., Fab or a peptide fragment. For example, an OKT3 antibody or OKT3 antibody fragment may be used.

Another major area for targeted delivery preferably involves the interleukin-2 (IL-2) system. IL-2 is a T-cell growth factor generally produced following antigen- or mitogen-induced stimulation of lymphoid cells. Cell types that typically produce IL-2 include, for example, CD4+ and CD8+ T-cells and large granular lymphocytes, as well as certain T-cell tumors. Generally speaking, IL-2 receptors are glycoproteins that are expressed on responsive cells. They are notable in connection with the present invention because they are generally readily endocytosed into lysosomal inclusions when bound to IL-2.

In addition to IL-2 receptors, preferred targets include the anti-IL-2 receptor antibody, natural IL-2 and an IL-2 fragment of a 20-mer peptide or smaller generated by phage display that binds to the IL-2 receptor. In use, for example, IL-2 may be conjugated to stabilizing materials, for example, in the form of vesicles, and thus may mediate the targeting of cells bearing IL-2 receptors. Endocytosis of the ligand-receptor complex may then deliver the compound to be delivered to the targeted cell. Additionally, an IL-2 peptide fragment which has binding affinity for IL-2 receptors may be incorporated, for example, by attachment to the termini of a different outer arm either directly to a reactive moiety or via a spacer or linker molecule with a reactive end such as an amine, hydroxyl, or carboxylic acid functional group. Such linkers are well known in the art and may comprise from 3 to 20 amino acid residues. In addition, D-amino acids or derivatized amino acids may be used which avoids proteolysis in the target tissue.

Still other systems which may be used in the present invention include IgM-mediated endocytosis in B-cells or a variant of the ligand-receptor interactions described above wherein the T-cell receptor is CD2 and the ligand is lymphocyte function-associated antigen 3 (LFA-3), as described, for example, in Wallner et al. (1987) J. Experimental Med. 166:923-932. Targeting ligands derived or modified from human leukocyte origin, such as CD11a/CD18 and leukocyte cell surface glycoprotein (LFA-1), may also be used as these may bind to the endothelial cell receptor ICAM-1. The cytokine inducible member of the immunoglobulin superfamily, VCAM-1, which is mononuclear leukocyte-selective, may also be used as a targeting ligand. VLA-4, derived from human monocytes, may be used to target VCAM-1.

Preferred targeting ligands in accordance with the present invention include, for example, Sialyl Lewis X, mucin, hyaluronic acid, LFA-1, N-formal peptide, C5a, leukotriene B4, platelet activating factor, IL-8/NAP-1, CTAP-III, RANTES, and 1-309. In addition, the integrins may be used as targeting ligands for targeting VLA-4, fibrinogen, von Willebrand factor, fibronectin, vitronectin, VCAM-1 and CD49d/CD29. A particularly preferred targeting ligand may be Sialyl Lewis X which binds to P-selectin and which has the following sequence: αNeu5Ac(2→3)βGal(1→4)[αFuc(1→3)]-βGlcNAc-OR wherein R is an aglycone having at least one carbon atom. P-selectin may be a preferred target because it typically localizes on the luminal side of endothelium during inflammation, but generally not in non-inflammatory synovia where it is generally cytoplasmic only.

Other preferred targeting ligands include, for example, antibodies directed to autoantigens on T-cell receptors. Peptides can be used to produce antibodies which may bind to the autoantigen portions of T-cell receptors. In addition, antibodies to additional T-cell and B-cell receptors may be used as targeting ligands. T- and B-cell receptors involved in inflammation and rheumatoid arthritis are described in Struyk et al. (1995) Arthritis & Rheumatism 38:577-89; Marchalonis et al. (1994) Immunobiol. Proteins Peptides, Plenum Press, New York, pp. 135-45; Dedeoglu et al. (1993) Immunol. Res. 12:12-20; Marchalonis et al. (1993) Exp. Clin. Immunogenet. 10:1-15; Dehghanpisheh et al. (1996) Immunological Invest. 25:241-52; Schluter et al. (1995) Immunobiol. Proteins Peptides VIII, Plenum Press, New York, pp.231-36; Lake et al. (1995) Immunobiol. Proteins Peptides VIII, Plenum Press, New York, pp.223-29; Lake et al. (1994) Biochem. Biophys. Res. Comm., 201:1502-1509; Lanchbury et al. (1995) British Med. Bull. 51:346-58; Marchaloniis et al. (1994) Autoantibodies Immunoglobulins T Cell Receptors., 129-47; Marchalonis et al. (1993) Gerentology., 39:65-79; Marchalonis et al. (1992) Proc. Natl. Acad. Sci. USA 89:3325-29; Sakkas et al. (1994) Immunol. Res. 13:117-38; Theofilpooulos et al. (1989) Springer Semin Immunopathol. 11:335-68; Cronstein et al. (1994) Curr. Opin. Rheum. 6:300-304; Szekanecz et al. (1996) J. Invest. Med., 44:124-135; Liao et al. (1995) Rheum. Arth. 21:715-740; Veale et al. (1996) Drugs & Aging 9:87-92; Cronstein et al. (1994) Curr. Opin. Rheum. 6:300-304; Haskard (1995) Curr. Opin. Rheum. 7:229-234; Cronstein et al. (1994) Curr. Opin. Rheum. 6:300-304; Remy et al. (1995) Proc. Natl. Acad. Sci. USA 92:1744-1748; Ashkenas et al. (1996) Dev. Biol. 180:433-444; Springer (1994) Cell 76:301-314; Hynes (1992) Cell 69:11-25; and Schwartz et al. (1995) Annu. Rev. Cell Dev. Biol. 11:549-599.

Additional targeting ligands which may be employed are described in Schwarzenberger et al. (1996) Blood. 87:472-8; Prokopova et al. (1993) J. Rheumatol. 20:673-7; U.S. Pat. No. 5,627,263 to Ruoslahti et al.; Chen et al. (1987) J. Cell Biol 105:1885-92; and Wallner et al. (1987) J. Exp. Med. 166:923-32.

Still additional targeting ligands that may be employed in the compositions and methods of the present invention are described in WO 96/37194 to Sullican et al., and include fetuin and asialofetuin, hexamine, spermine and spermidine, N-glutaryl DOPE, IgA, IgM, IgG and IgD, MHC and HLA markers, and CD1, CD4, CD8-11, CD15, Cdw17, CD18, CD21-25, CD27, CD30-45, CD46-48, Cdw49, Cdw50, CD51, CD53-54, Cdw60, CD61-64, Cdw65, CD66-69, Cdw70, CD71, CD73-74, Cdw75, CD76-77, LAMP-1 and LAMP-2.

Exemplary covalent bonds through which the targeting ligands may be covalently linked to the termini of the outer arms include, for example, amide (—CONH—); thioamide (—CSNH—); ether (ROR′, where R and R′ may be the same or different and are other than hydrogen); ester (—COO—); thioesters (—COS—); —O—; —S—; —Sn—, where n is greater than 1, preferably about 2 to about 8, and more preferably about 2; carbamates; —NH—; —NR—, where R is alkyl, for example, alkyl of from 1 to about 4 carbons; urethane; substituted imidate; and combinations of two or more of these. Covalent bonds between targeting ligands and stabilizing materials, for example, lipids, may be achieved through the use of molecules that may act as spacers to increase the conformational and topographical flexibility of the ligand. Examples of such spacers include, for example, succinic acid, 1,6-hexanedioic acid, 1,8-octanedioic acid, and the like, as well as modified amino acids, such as, for example, 6-aminohexanoic acid, 4-aminobutanoic acid, and the like. In addition, in the case of targeting ligands that comprise peptide moieties, sidechain-to-sidechain crosslinking may be complemented with sidechain-to-end crosslinking and/or end-to-end crosslinking. Also, small spacer molecules, such as dimethylsuberimidate, may be used to accomplish similar objectives. The use of agents, including those used in Schiffs base-type reactions, such as gluteraldehyde, may also be employed. The Schiffs base linkages, which may be reversible linkages, can be rendered more permanent covalent linkages via the use of reductive amination procedures. This may involve, for example, chemical reducing agents, such as lithium aluminum hydride reducing agents or their milder analogs, including lithium aluminum diisobutyl hydride (DIBAL), sodium borohydride (NaBH₄) or sodium cyanoborohydride (NaBH₃CN).

The covalent linking of the targeting ligands to the stabilizing materials in the present compositions may be accomplished using synthetic organic techniques that would be readily apparent to one of ordinary skill in the art in view of the present disclosure. For example, the targeting ligands may be linked to the materials via the use of well-known coupling or activation agents. As known to the skilled artisan, activating agents are generally electrophilic, which can be employed to elicit the formation of a covalent bond. Exemplary activating agents that may be used include, for example, carbonyldiimidazole (CDI), dicyclohexylcarbodiimide (DCC), diisopropylcarbodiimide (DIC), methyl sulfonyl chloride, Castro's Reagent, and diphenyl phosphoryl chloride.

The covalent bonds may involve crosslinking and/or polymerization. Crosslinking preferably refers to the attachment of two chains of polymer molecules by bridges, composed of an element, a group, or a compound, which join certain carbon atoms of the chains by covalent chemical bonds. For example, crosslinking may occur in polypeptides that are joined by the disulfide bonds of the cysteine residue. Crosslinking may be achieved by any number of methods including the addition of a chemical substance (crosslinking agent) and exposing the mixture to heat, and the exposure of the polymer to high energy radiation. A variety of crosslinking agents, or “tethers,” of different lengths and/or functionalities are described, for example, in R. L. Lunbland (1995) Techniques in Protein Modification, CRC Press, Inc., Ann Arbor, Mich., pp. 249-68. Exemplary crosslinkers include, for example, 3,3′-dithiobis(succinimidylpropionate), dimethyl suberimidate, and its variations thereof, based on hydrocarbon length, and bis-N-maleimido-1,8-octane.

The targeting ligands may be linked or attached via a linking group. Preferably, the targeting ligand is attached via a linker that is also attached to the arms of the polymer. A variety of linking groups are available and would be apparent to one skilled in the art in view of the present disclosure. Preferably, the linking group comprises a hydrophilic polymer. Suitable hydrophilic linker polymers include, for example, polyalkyleneoxides such as, for example, PEG and polypropylene glycol (PPG), polyvinylpyrrolidones, polyvinylmethylethers, polyacrylamides, such as, for example, polymethacrylamides, polydimethylacrylamides and polyhydroxypropylmethacrylamides, polyhydroxyethyl acrylates, polyhydroxypropyl methacrylates, polymethyloxazolines, polyethyloxazolines, polyhydroxyethyloxazolines, polyhyhydroxypropyloxazolines, polyvinyl alcohols, polyphosphazenes, poly(hydroxyalkylcarboxylic acids), polyoxazolidines, polyaspartamide, and polymers of sialic acid (polysialics). The hydrophilic polymers are preferably selected from the group consisting of PEG, PPG, polyvinylalcohol and polyvinylpyrrolidone and copolymers thereof, with PEG and PPG polymers being more preferred and PEG polymers being even more preferred. Preferred among the PEG polymers are, for example, bifunctional PEG having a molecular weight of about 1,000 daltons to about 10,000 daltons, preferably about 5,000 daltons. Preferably, the polymer is bifunctional with the targeting ligand bound to a terminus of the polymer. Generally, the targeting ligand may be incorporated into the stabilizing agent at concentrations of from about 0.1 to about 25 mole %, preferably from about 1 to about 10 mole %. Of course, the particular ratio employed may depend upon the particular targeting ligand, linker group, and stabilizing agents.

Standard peptide methodology may be used to link the targeting ligand to the stabilizing materials when utilizing linker groups having two unique terminal functional groups. Bifunctional hydrophilic polymers, and especially bifunctional PEGs, may be synthesized using standard organic synthetic methodologies. In addition, many of these materials are available commercially, such as, for example, “-amino-T-carboxy-PEG which is commercially available from Shearwater Polymers (Huntsville, Ala.). An advantage of using a PEG material as the linking group is that the size of the PEG may be varied such that the number of monomeric subunits of ethylene glycol may be as few as about 5, or as many as about 500 or even greater. Accordingly, the “tether” or length of the linkage may be varied, as desired. This may be important depending on the particular targeting ligand employed. For example, a targeting ligand that comprises a large protein molecule may require a short tether, and thereby simulate a membrane-bound protein. A short tether may also allow for a delivery polymer to maintain a close proximity to the target. This may be used advantageously in connection with vesicles that also comprise an active agent in that the concentration of active agent that may be delivered to the cell may be advantageously increased. Another suitable linking group that may provide a short tether is glyceraldehyde. Glyceraldehyde may be bound to DPPE via a Schiff's base reaction. Subsequent Amadori rearrangement can provide a substantially short linking group. The gamma carbonyl of the Schiffs base may then react with a lysine or arginine of the targeting protein or peptide to form the targeted lipid.

In certain embodiments, the targeting ligands, may be incorporated in the polymer stabilizing agent via non-covalent associations. As known to those skilled in the art, non-covalent association is generally a function of a variety of factors, including, for example, the polarity of the involved molecules, the charge (positive or negative), if any, of the involved molecules, the extent of hydrogen bonding through the molecular network, and the like. Non-covalent bonds are preferably selected from the group consisting of ionic interaction, dipole-dipole interaction, hydrogen bonds, hydrophilic interactions, van der Waal's forces, and any combinations thereof. Non-covalent interactions may be employed to bind the targeting ligand to the stabilizing agent. Additional techniques that may be adapted for incorporating the targeting ligand into the present compositions are disclosed, for example, in U.S. Pat. No. 6,521,211 to Unger et al.

D. Excipients, Secondary Stabilizing Agents, and Other Components of the Formulation

Other moieties may be incorporated into the present formulations as excipients in order to reduce the particle size of the stabilized drug matrix.

Suitable excipients include free phospholipids, which may or may not be the same as the phospholipid moieties conjugated to the hydrophilic polymer. These free phospholipids, i.e., phospholipids not conjugated to PEG or other moieties, may be incorporated into the present formulations as excipients in order to reduce the particle size of the polymer/drug matrix. The free phospholipid, like the phospholipid that may be conjugated to the hydrophilic polymer, can be anionic, neutral or cationic, of naturally occurring or synthetic origin, and will generally comprise a diacyl phosphatidylcholine, a diacyl phosphatidylethanolamine, a diacyl phosphatidylserine, a diacyl phosphatidylinositol, a diacyl phosphatidylglycerol or a diacyl phosphatidic acid, wherein each acyl moiety can be saturated or unsaturated and typically contains about 8 to 20 carbon atoms. As with the conjugated phospholipids, the preferred unsaturated acyl moieties of the free phospholipids are oleic and linoleic acid esters, and preferred saturated acyl moieties are palmitate, myristate and stearate; particularly preferred phospholipids are DPPE and POPE. The amount of free phospholipid should be just sufficient to reduce the particle size as desired. Preferably, any free phospholipid that is included in the formulation represents less than about 25% of the total phospholipid present, and optimally represents less than about 10% of the total phospholipid present.

Compounds other than free phospholipids are also useful for reducing particle size, and can be used in addition to or in lieu of free phospholipids; these other compounds include, but are not limited to, cholic acids, cholic acid salts, saccharides (such as sorbitol, sucrose, trehalose, mannitol, inositol), polyhydroxyalcohols (such as glycerol), and liquid polyethylene glycols (i.e., PEG having a molecular weight less than about 1000 daltons). The formulations of the invention can also contain pharmaceutically acceptable auxiliary agents as required in order to approximate physiological conditions; such auxiliary agents include pH adjusting and buffering agents (e.g., citrate and phosphate buffers), tonicity adjusting agents, and the like. Lipid-protecting agents that serve to minimize free radical and peroxidative damage upon storage may also be advantageous. Suitable lipid protective agents include alpha-tocopherol, ethylenediaminetetraacetic acid (EDTA), and water-soluble, iron-specific chelators such as deferoxamine. Additionally, for lyophilized compositions that are to be hydrated prior to use, it may be desirable to include one or more cryoprotectants, or anti-flocculants in order to facilitate rehydration and formation of a substantially homogeneous suspension. For compositions that are to be stored in liquid form, it is preferred that one or more conventional anti-bacterial agents be included. Still other additives that may be incorporated into the present formulations include radioactive or fluorescent markers useful for imaging purposes. Radioactive markers include, for example, technetium-99 and indium-111, while an exemplary fluorescent marker is fluorescein. The excipients can be included in an amount up to about 50 wt % of the formulation, but preferably represent less than about 10 wt % of the formulation.

Secondary stabilizing agents may also be added to the formulation and are useful for reducing particle size. These agents are typically polymers or proteins and be used in addition or in lieu of free phospholipids. Preferably, the secondary stabilizing agent acts to stabilize the surface of the complex by virtue of a combination of hydrophilic and hydrophobic interactions. Thus, it is preferred that the secondary stabilizing agent polymer contains both hydrophilic and hydrophobic groups or domains thus allowing this interaction to occur. It is also preferred that the secondary stabilizing agent contain a sufficient amount of hydrophilic surfaces that post-stabilization nanoparticles remain suspended within an aqueous solution and avoid clumping.

An exemplary secondary stabilizing agent is a polymer having a molecular weight ranging from about 400 daltons to about 400,000 daltons, more preferably from about 1,000 daltons to about 200,000 daltons, and still more preferably from about 3,000 daltons to about 100,000 daltons. The secondary stabilizing agent may be derived from natural, recombinant, synthetic or semisynthetic sources. Most preferably the stabilizing agent will be a protein or a peptide. Useful preferred proteins include albumin, collagen, fribrin, immunoglobulins, hemoglobin, vascular endothelial growth factor, vascular permeability factor, epidermal growth factor, fibroblast growth factor, fibronectin, vitronectin, and cytokines such as interleukins (e.g. IL-3 and IL-12).

Suitable proteins include, but are not limited to: serum proteins, i.e., albumin (especially recombinant and defatted), amylins, atrial natriuretic peptides, endothelins and endothelin inhibitors, urokinase, streptokinase, staphylokiase, vasoactive intestinal peptide, HDL, LDL, VLDL, etc.; agglutination (antihemophillia) factors, i.e., Factor VIII, Factor IX and subtypes thereof, decorsin, serum thymic factor, etc.; peptide hormones, i.e., ACTH, FSH, LH, parathyroid hormone, thyroxin, insulin, vasopressin, bradykinin and bradykinin potentiators, HGH, CRF (corticotropin releasing factor), oxytocin, gastrins, LH-RH, MSH (melanocyte stimulating hormone) and MSH releasing factor; parathyroid hormones and analogs; pituitary adenylate cyclase activating polypeptide; secretins; thyrotropin releasing hormone, etc.; structural proteins, i.e., collagens, amyloid proteins, brain natriuretic peptides, elafin, fibronectin and fibronectin fragments, laminin, sarafotoxins, etc.; growth factors, i.e., nerve growth factor, platelet derived growth factor, epidermal growth factor, vascular endothelial growth factor, tumor necrosis factor, CINC-I (cytokine-induced neutrophil chemoattractant), growth hormone releasing factor, liver cell growth factor, midkines, neurokinins, neuromedins, etc., metabolic potentiators, i.e., erythropoietin, adrenomedullin and adrenomedullin antagonists, (o-agatoxin TK, agelenin, angiotensins, calcicludine, calciseptine, calcitonin and calcitonin antagonists, calmodulin, charybdotoxin, chlorotoxin, conotoxins, endorphins, neo-endorphins, glucagon and variants, guanylins, iberiotoxin, kaliotoxin, margatoxin, mast cell degranulating peptide, neurotensins, pancreastatins, PLTX-11, scylotoxin, ATPase inhibitors, somatostatins, somatomedin, uroguanylin, etc.; nuclear binding proteins, i.e., histones, spermine, spermidine, nuclear localization sequences, telomerase, etc.; enzymes, i.e., cholecystokinin, cathepsins, etc.; antivirals, i.e., IFN-α, IFN-β, IFN-γ, virus replication inhibiting peptide, etc.; immunoglobulins, i.e., IgA, IgD, IgE, IgG, IgH and subtypes; and miscellaneous proteins such as apamin, bombesin, casomorphins, conantokins, defensin-1, dynorphins, enkephalins, galanins, magainin, nociceptin, osteocalcins, substance P, xenin, etc. While not wishing to be limited to the preceding examples, one of skill in the art will recognize that the examples given may be used individually or in combination.

The secondary stabilizing protein may also serve as a targeting agent or binding ligand to direct the nanoparticles and drugs therein to a certain site. One preferred protein is albumin, in particular, human serum albumin and even more preferably recombinant derived human albumin. Another preferred protein is defatted albumin, either native or recombinant. For veterinary applications, the albumin is preferably from the patient's species. The stabilizing albumin is generally added to the nanoparticles at an effective stabilizing concentration, generally in the range of about 0.001 to up to about 10% w/v, more preferably in the range of about 0.01 to about 5%, in the range of about 0.1 to about 2.5%, and most preferably in the range of about 0.25 to about 1.5%. Note that more than one protein may be used to stabilize the nanoparticles. For example, the particles may be formulated with about 1.0% w/v albumin and about 0.1% w/v EGF. In this case, the EGF serves as a targeting ligand to help the nanoparticle to bind to tissues with increased expression of the EGF receptor.

The protein may be naturally occurring, a protein fragment, e.g. a fragment of the gamma-carboxy terminus of fibrinogen, or chemically modified. For example, albumin or other proteins may be modified with one or more hydrophilic or targeting moieties. For example, the protein may be modified by binding one or more PEG residues per protein molecule, typically between 1 and 100 PEG molecules per protein molecule, but more preferably between 1 and 10 PEG residues. For example, mono or bifunctional PEG groups may be coupled to the protein through linkages such as ethers or biodegradable bonds such as esters, amides, carbamates, thioesters, disulfides, thiocarbamates, phosphate esters and phosphoamides. The resulting “PEGylated” protein enables the protein to stabilize the surface of the nanoparticle while the PEG groups help to protect the nanoparticle surface from nonspecific interaction with serum proteins. In this regard, the “PEGylated” proteins increase the serum half-lives of the nanoparticles.

The secondary stabilizing agent may also be a natural polymer, such as: cellulose and dextran; semi-synthetic cellulose derivatives such as methylcellulose and carboxymethyl cellulose; and synthetic polymers such as polyinylalcohol polyvinylpyrrolidone and copolymers containing PEG and a second polymer such as polypropylene glycol (PPG) (e.g. those available under the Pluronic trademark). Synthetic polymers such as the Pluronics, i.e. copolymers of PEG and PPG, may be incorporated into mixtures of stabilizing agents, e.g., with albumin. Preferred block copolymers include, but are not limited to, polyethylene glycol-N-carboxyanhydride of 6-(benzyloxycarbonyl)-1-lysine, polyethylene glycol-poly-1-lysine and polyethlyeneglycol-polyaspartic acid. Methods for synthesizing the above copolymers are detailed in Harada et al (1995) Macromolecules 28:5294-5299. One of skill in the art will readily recognize that the same synthetic methods can be used to substitute polypropylene glycols for PEG to make the PPG block copolymer analogs of the above.

E. Acoustically Active Gas

In a further embodiment of the invention, the present formulations are made with small quantities of an acoustically active gas instilled therein. In order to instill the selected gas into the present formulations, a headspace of gas (preferably an insoluble gas) is applied atop the lyophilized composition in a closed container, which is then exposed to mild agitation during rehydration. Microquantities of gas will become entrapped in the interstices of the dispersion. The presence of the acoustically active gas is useful in conjunction with ultrasound imaging, as the gas-instilled dispersion produces an echogenic contrast that allows the drug to be tracked in the body. In addition, if a sufficient quantity of gas is entrapped in the formulation, therapeutic ultrasound can allow the microstructure to unfold at the locus where the ultrasound is applied, releasing the active agent and thus enhancing targeting effectiveness. The acoustically active gas lowers the cavitation threshold, i.e., the energy required for cavitation with ultrasound. Preferably, the cavitation energy used will be under about 1.5 MegaPascals, and more preferably under about 1.0 MegaPascals. The gas also effects dB reflectivity, and a gas concentration of about 1 mg per ml of particles will generally have a reflectivity approximately 2 dB higher than that of pure water.

In general, the amount of acoustically active gas that is imbibed by the particles of the formulation is approximately equal to the void space within the particles, which can be approximated by their density. For example, particles having a density of 0.10 will imbibe about 90 vol. % gas. Lower density particles will imbibe a higher volume of gas (i.e., 95 vol. % for particles having a density of 0.05), while higher density particles will imbibe a lower volume of gas (i.e., 85 vol. % for particles having a density of 0.15). Gas may also adhere to the surface of the particles, typically up to about two times the volume of the particles. Normally, the amount of acoustically active gas that is employed is such that the gas-instilled formulation will contain at least about 5 vol. % gas, preferably about 10-15 vol. % gas.

Typical acoustically active gases are chemically inert gases having 1 to 12 carbon atoms, and particularly preferred acoustically active gases are perfluorocarbons, including saturated perfluorocarbons, unsaturated perfluorocarbons, and cyclic perfluorocarbons. The saturated perfluorocarbons, which are usually preferred, have the formula CₙF₂ₙ₊₂, where n is from 1 to 12, preferably 2 to 10, more preferably 4 to 8, and most preferably 5. Examples of suitable saturated perfluorocarbons are the following: tetrafluoromethane; hexafluoroethane; octafluoropropane; decafluorobutane; dodecafluoropentane; perfluorohexane; and perfluoroheptane. Saturated cyclic perfluorocarbons, which have the formula CₙF₂ₙ, where n is from 3 to 8, preferably 3 to 6, may also be preferred, and include, e.g., hexafluorocyclopropane, octafluorocyclobutane, and decafluorocyclopentane. Other gases that can be used include air, nitrogen, helium, argon, xenon and other such gases.

Alternatively, a gaseous precursor can be used that is in the liquid state at room temperature and that is either (1) volatilized prior to introduction into the headspace above the lipid- and drug-containing dispersion, or (2) volatilized and instilled into a microemulsion which is then introduced into the lipid- and drug-containing dispersion. Suitable gaseous precursors are described, for example, in U.S. Pat. No. 5,922,304 to Unger, and include, without limitation: hexafluoro acetone, isopropyl acetylene, allene, tetrafluoroallene, boron trifluoride, isobutane, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluoro-butane, 2-methyl-butane, decafluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluorobutyne, 3-methyl-1-butyne, perfluoro-2-butyne, 2-bromobutyraldehyde, carbonyl sulfide, crotononitrile, cyclobutane, methyl-cyclobutane, octafluorocyclobutane, perfluorocyclobutene, 3-chlorocyclopentene, octafluorocyclopentenecyclopropane, 1,2-dimethyl-cyclopropane, 1,1-dimethylcyclopropane, 1,2-dimethylcyclopropane, ethylcyclopropane, methylcyclopropane, diacetylene, 3-ethyl-3-methyl diaziridine, 1,1,1-trifluorodiazoethane, dimethyl amine, hexafluorodimethylamine, dimethylethylamine, bis(dimethylphosphine)amine, perfluorohexane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoro-ethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropenta-fluoroethane, dichlorotrifluoroethane, fluoroethane, hexafluoroethane, nitropentafluoroethane, nitrosopentafluoroethane, perfluoroethylamine, ethyl vinyl ether, 1,1-dichloroethane, 1,1 -dichloro-1,2-difluoroethane, 1,2-difluoroethane, methane, trifluoromethanesulfonylchloride, trifluoromethane-sulfonylfluoride, bromodifluoronitrosomethane, bromofluoromethane, bromochloro-fluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodo-methane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsulfide, methyl vinyl ether, neon, neopentane, nitrogen (N₂), nitrous oxide, 1,2,3-nonadecane-tricarboxylic acid-2-hydroxytrimethylester, 1-nonene-3-yne, oxygen (O₂), 1,4-pentadiene, n-pentane, perfluoropentane, 4-amino-4-methylpentan-2-one, 1-pentene, 2-pentene (cis), 2-pentene (trans), 3-bromopent-1-ene, perfluoropent-1-ene, tetrachlorophthalic acid, 2,3,6-trimethylpiperidine, propane, 1,1,1,2,2,3-hexafluoropropane, 1,2-epoxypropane, 2,2-difluoropropane, 2-aminopropane, 2-chloropropane, heptafluoro-1-nitropropane, heptafluoro-1-nitrosopropane, perfluoropropane, propene, hexafluoropropane, 1,1,1,2,3,3-hexa-fluoro-2,3 dichloropropane, 1-chloropropane, chloropropane (trans), 2-chloropropane, 3-fluoropropane, propyne, 3,3,3-trifluoropropyne, 3-fluorostyrene, sulfur hexafluoride, sulfur (di)-decafluoride(S₂F₁₀), 2,4-diaminotoluene, trifluoroacetonitrile, trifluoromethyl peroxide, trifluoromethyl sulfide, tungsten hexafluoride, vinyl acetylene, vinyl ether, and xenon.

Additionally, ferromagnetic, paramagnetic or superparamagnetic iron may be incorporated into the particles. This may be accomplished by dissolving iron salts, e.g. ferrous sulfate and ferric chloride in acidic conditions and adding base to produce iron oxide particles (e.g. Fe₃O₄). When performed under agitation conditions (e.g. mechanical shearing, centrifugation, microemulsification and the like) this may preferentially form iron oxide nanoparticles under 100 nm diameter. The iron oxide nanoparticles may be incorporated into the drug delivery nanoparticles which are preformed or as part of the process of manufacturing the particles themselves. In so doing magnetic drug delivery nanoparticles can be produced. These may be used with magnetic fields to do site specific drug delivery, the magnetic fields being employed to localize drugs within a certain area, e.g. tumors or the brain. Similarly optically active magnetic materials such as porphyrins can be incorporated into the nanoparticles so that they may be locally activated with light energy.

Because the preferred particles in this invention have an anisotropic morphology, i.e. they are much longer that they are wide, they have very unusual properties for drug delivery. Typical SN-38 particles of this invention may have a diameter of about 20 to 50 nm and a length of 500 nm to 1 micron. It is believed that, after vascular administration, the particles flow through the body via laminar flow, generally with their long axes oriented parallel to the direction of blood flow. Upon entering a diseased tissue, such as a tumor, the particles encounter the irregular chaotic flow of tumor vessels. It is believed that the anisotropic nature of the particles is useful for drug delivery. As the nanoparticles enter the chaotic microvessels of the tumor, their axes may shift and they may become lodged sidewise within the tumor microvessels. This propensity may increase delivery of drug to the diseased tissue. For energy-mediated drug delivery, i.e. when energy is focused or applied on the diseased tissue, these anisotropic nanoparticles may be very useful. Ultrasound can be used to “flip” the particles so that they lodge sidewise into the tissue. The long axis of the particles, creates a highly magnetic material, when the particles are magnetized, so that they are more readily manipulated by the magnetic field. Light energy is also more favorably absorbed by the particles when such energy is applied to encounter as a wave the long axes of the particles.

V. Utility

The nanoparticles produced by the methods described herein are biocompatible and highly useful for drug delivery. The drug delivery is preferably via IV injection but the technology has applications for oral, subcutaneous, e.g., sustained release, and pulmonary delivery. For IV delivery, the nanoparticles decrease toxicity of the therapeutic agents such as paclitaxel. Larger doses of active agents can therefore be administered via IV, allowing for higher blood levels of the therapeutic agent yielding greater efficacy. For oral applications, the nanoparticles improve dispersal of insoluble drugs and increase uptake from the gastrointestinal tract. For sustained release applications, the nanoparticles can be formulated into gels, powders or suspensions. For pulmonary applications, the nanoparticles' small effective hydrodynamic radii improves delivery of therapeutic agents into the distal airways, such as the alveoli, thereby allowing systemic delivery of active agents via the pulmonary route.

The size of the particles within the formulation helps to control dispersal of the drug and drug release. Surprisingly, stabilized and unstabilized drug/polymer complexes have improved solubility and drug release properties compared to crystalline forms of the drug. The rate of release of drug/polymer complexes can be fine-tuned by optionally including a stabilizing agent and by varying the nature of the drug complex. For example, branched polyethylene glycol (PEG) is a soluble polymer that is capable of forming complexes with certain hydrophobic drugs. Once in the body, the PEG will eventually dissolve, releasing the complexed drug. The rate of drug release can be modified by varying the conditions and parameters of complex formation, e.g., ratios of PEG to drug, chemical structure of the PEG, and the amount and type of stabilizing agent. Hydrolyzable bonds may also be incorporated into the hydrophilic polymer and/or the stabilizing agent to accelerate drug release, and pH-responsive groups may be used to increase drug release at a desired pH.

The formulations made by the methods of the invention are useful to treat a mammalian individual, generally a human patient, suffering from a condition, disease or disorder that is responsive to systemic administration of a particular drug. The formulations may be administered orally, parenterally, topically, transdermally, rectally, vaginally, by inhalation, intra-ocularly, in an implanted reservoir (i.e., in a sustained release depot for subcutaneous or intramuscular administration), or as a packing material for wounds and fractures. The term l“parenteral” as used herein is intended to include subcutaneous, intravenous, intramuscular, intra-arterial, intrathecal and intraperitoneal injection, and the formulation may be injected as either a bolus or an infusion. Since the invention provides formulations that substantially increase the systemic bioavailability of a drug having low aqueous solubility, dosages can be significantly reduced relative to that used in conjunction with conventional pharmaceutical compositions containing the same active agent. Analogously, a conventional dosage or an increased dosage can be used to provide substantially higher blood levels of a drug than previously obtained using conventional formulations. For paclitaxel, by way of example, a bolus dosage of at least 3.5 mg/kg and even 7.0 mg/kg can be administered using the formulations made by the methods described herein, while with continuous infusion, a dosage of at least 140 mg/kg can be administered and with using a stabilized formulation, a dosage of up to 200 mg/kg can be administered.

The formulations made by the methods of the invention can be also be used so that a drug is targeted to a particular cell type or tissue. In this embodiment, a targeting agent is employed that is covalently coupled to the stabilizing agent such as through a terminal hydroxyl group of polyethylene glycol. Suitable targeting agents are those that are generally used with liposomal formulations, e.g., peptides, peptide fragments, antibodies or peptidomimetics, although other ligands such as saccharides and folates can also be used. Preferred targeting agents are integrins such as the β₃ integrins (“cytoadhesins”), with cyclized oligopeptides containing the Arg-Gly-Asp (RGD) motif particularly preferred.

The present formulations are also useful as packing materials for wounds and fractures, and as coating materials for endoprostheses such as stents, grafts and joint prostheses. It is known that restenosis (narrowing of the blood vessels) may occur after angioplasty, placement of a stent, and/or other coronary intervention procedures, as a result of fibroblast proliferation and smooth muscle hypertrophy. Thus, the formulations of the invention may be used as coating materials for endoprostheses to provide local drug delivery following coronary intervention.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entirety.

### EXPERIMENTAL

The following examples are put forth so as to provide those of ordinary skill in the art with a complete disclosure and description of how to prepare and use the formulations disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. All materials were purchased from commercial sources such as Polymer Source (Dorval, Canada), Avanti Polar Lipids (Alabaster, AL), Genzyme Pharmaceuticals (Cambridge, Mass.), or Northern Lipids (Vancouver, British Columbia). All other materials were obtained as follows: SN-38 (Decode Genetics, Woodbridge, Ill.); paclitaxel (Natural Pharmaceuticals, Inc., Beverly, Mass.); poloxamer (Poloxamer 188, BASF, Parsippany, N.J.); mannitol (Aldrich Chemical Company, Milwaukee, Wis.); and sorbitol (Fischer Chemical, Fairlawn, N.J.).

### Example 1

### Formulation of SN-38

SN-38 (5 mg/mL) and sorbitol (100 mg/mL) were dissolved in warm DMSO. 6.75 mL of this solution was combined with a mixture of 900 mg POPC, 45 mg DOPG, 22.5 mg DOPE-PEG 5000, and 450 mg PEG 600 in 128.25 mL of tert-butyl alcohol (t-butanol). The solution was filtered with a sterilizing filter and aliquoted into vials. The vials were placed in a lyophilizer and lyophilized according to a standard cycle. The resulting powder was rehydrated with purified water and sonicated for 60 seconds. The particle size of the resulting suspension was approximately 300 nm.

### Example 2

### Formulation of SN-38

SN-38 (2 g) and sorbitol (15 g) were dissolved in 150 mL of DMSO. 20 g of POPC, Ig DOPG, 0.5 g DOPE-PEG 5000, and 10 g 6K linear PEG were dissolved in 850 mL of t-butanol in a separate beaker. The solutions were heated to no more than 75° C. to dissolve the components. The DMSO and the t-butanol solutions were combined, and stirred to mix. The resulting solution was sterile-filtered through a 0.2 μm nylon filter, and 9-mL aliquots were filled into 20 cc vials. The vials were stoppered in the lyo-position and lyophilized using a standard cycle.

| concentrations | mg/mL | molar | ratio to SN-38 |
|---|---|---|---|
| SN-38 | 1.5 | 0.004 | NA |
| POPC | 5 | 0.007 | 1.72:1 |
| DOPG | 1 | 0.001 | 0.33:1 |
| POPE-PEG | 20 | 0.007 | 1.88:1 |
| Sorbitol | 5 | 0.027 | 7.18:1 |

### Example 3

### Formulation of Paclitaxel

10K 4-arm branched-PEG (6.5126 g, 50 mg/mL), POPG (2.6219 g, 20 mg/mL) and paclitaxel (1.3040 g, 10 mg/mL) were weighed out and put in a 500 mL round-bottom flask. T-butanol (260 mL) was added to the flask followed by heating at 60° C. for 90 min. The mixture was sonicated for 5 min then heated for another 30 min. All solids were completely dissolved. Vials were previously autoclaved and opened in a clean bio-safety cabinet. The solution was filtered through a VacuCap filter with a Supor membrane 0.8/0.2 μm. Solution was divided into 20 batches of 12 mL and placed in 20 mL glass vials. All vials were lyophilized overnight. Vials were capped under argon atmosphere followed by placement of crimp cap on each vial.

Diluent was prepared by mixing 5.0068 g of Myrj-52 (20 mg/mL) with 250 mL of purified water. The solution was filtered through a VacuCap filter with a Supor membrane 0.8/0.2 μm in a clean bio-safety cabinet. Each vial was hydrated with 5 mL of diluent (20 mg/mL Myrj-52) and vortexed for 1 min. followed by sonication for 5 min. Very few particles were visible under normal light (<1 μm) and no particles were visible under polarized light.

### Example 4

### Formulations of SN-38

All the ingredients were weighed out into a round bottom flask. The ingredients were dissolved in 5% DMSO/95% t-butanol. In order to solubilize the excipients, the fill volume used in each vial was 3 times the required hydration volume. The solution was filtered through a 0.2 μm DMSO-safe filter directly into sterile vials. The vials were frozen at −50° C. for 2 hrs, followed by a lyophilization cycle at −5° C. for 16 hrs and a secondary drying cycle at 40° C. for 6 hrs. The vials containing dry powder were then sealed under a partial vacuum.

Alternatively, all the excipients were dissolved in 95% tert-butanol, optionally heating to 75° C. and SN-38 was dissolved in 5% DMSO separately. The dissolved SN-38 was then added directly to the solution of excipients in tert-butanol and aliquoted into vials.

The following are some of the formulation compositions that have been prepared using these methods:

a) 10 mg/mL POPC;

0.5 mg/mL DOPE-PEG 5K;

25 mg/mL 6K linear PEG; and

1 mg/mL SN-38.

b) 10 mg/mL POPC;

0.5 mg/mL DOPE-PEG 5K;

25 mg/mL 6K linear PEG;

2.5 mg/mL Poloxamer; and

1 mg/mL SN-38.

c) 20 mg/mL POPC;

1 mg/mL DOPG;

25 mg/mL 10K linear PEG; and

1 mg/mL SN-38.

d) 2 mg/mL four-arm poly(ethylene oxide-b-α-caprolactone)

(branched PEG-b-polycaprolactone);

0.5 mg/mL DOPE-PEG 5K;

20 mg/mL POPC;

15 mg/mL 6K linear PEG; and

0.75 mg/mL SN-38.

e) 5 mg/mL branched PEG-b-polycaprolactone;

1 mg/mL DOPG; and

1 mg/mL SN-38.

f) 10 mg/mL DOPE-PEG 2k;

1 mg/mL DOPG;

5 mg/mL branched PEG-b-polycaprolactone;

5 mg/mL sorbitol; and

1.5 mg/mL SN-38.

g) 20 mg/mL sorbitol;

1 mg/mL DOPG;

5 mg/mL branched PEG-b-polycaprolactone; and

1.5 mg/mL SN-38.

### Example 5

### Formulation of SN-38

The SN-38, four-arm poly(ethylene oxide-b-ε-caprolactone), and DOPG were dissolved in an amount of DMSO equal to 5% of the final volume. A 1.2% (w/v) mannitol solution was prepared in an amount of water equal to 25% of the final volume. The mannitol solution was combined with an amount of tert-butyl alcohol equal to 70% of the final volume. The DMSO solution was combined with the mannitol/water/TBA solution and mixed thoroughly. The resulting clear solution was sterile filtered through a 0.2 μm filter and filled into 10 cc vials at a fill volume of 4.5 mL. The vials were loaded unto −45° C. shelves and lyophilized according to a standard cycle. The resulting lyophilized cake was easily hydrated with water for injection to yield a translucent solution which contained very little crystalline matter when observed under a light microscope. Electron microscopy showed that the particles have a rod-like structure that is 1 to 5 μm in length.

Optimal Ratios for the formulation:

| Concentrations* | mg/mL | molar | ratio to SN-38 |
|---|---|---|---|
| SN-38 | 1.5 | 0.004 | NA |
| Caprolactone | 10 | 0.001 | 0.22:1 |
| DOPG | 1 | 0.001 | 0.33:1 |
| Mannitol | 20 | 0.329 | 86.1:1 |

We claim:

1. A method of producing a sterile pharmaceutical formulation comprising:
(a) admixing, in an organic solvent, a drug, and a stabilizing agent that stabilizes the drug but does not covalently bind thereto; wherein the organic solvent has a freezing temperature with the range of about 0-25° C.;
(b) filter sterilizing the mixture; and
(c) removing the organic solvent in a manner effective to provide a dry formulation of the drug.

2. The method of claim 1, wherein the organic solvent is selected from tert-butyl alcohol, cyclohexane, dimethyl carbonate, dimethyl sulfoxide, and acetic acid.

3. The method of claim 1 wherein the admixing step further comprises including a second solvent.

4. The method of claim 3, wherein the second solvent is selected from alkylated alcohols, ethers, acetone, alkanes, dimethyl sulfoxide, chloroform, cyclic hydrocarbons, toluene, benzene, N,N-dimethylformamide, and mixtures thereof.

5. The method of claim 3, wherein the admixing step further comprises including a water-soluble bulking agent.

6. The method of claim 5, wherein the water-soluble bulking agent is selected from sorbitol, mannitol, xylitol, hydrogenated starch hydrolysates, maltitol, lactitol, maltitol, hydrogenated isomaltulose, erythritol, inositol, sucrose, and trehalose.

7. The method of claim 1, which further comprises the step of sonicating the formulation.

8. The method of claim 1, which further comprises the step of purging with an inert gas.

9. The method of claim 1, wherein the solvent is removed by lyophilization.

10. The method of claim 1, wherein the solvent is removed by spray drying.

11. The method of claim 1, wherein solvent removal step comprises rotary evaporation, thereby providing an agglomerated intermediate product, and wherein the method further comprises deagglomerating the intermediate product using a procedure effective to provide the dry formulation of the drug.

12. The method of claim 1, wherein the admixing step further comprises including a supercritical fluid.

13. The method of claim 12, wherein the solvent is removed by supercritical fluid processing.

14. The method of claim 1, wherein the formulation is particulate.

15. The method of claim 14, wherein the formulation is comprised of particles that have an average size in the range of approximately 1 nm to about 1000 μm.

16. The method of claim 15, wherein the average size of the particles is in the range of approximately 10 to 10,000 nm.

17. The method of claim 16, wherein the average size of the particles is in the range of approximately 50 to 1000 nm.

18. The method of claim 17, wherein the average size of the particles is in the range of approximately 200 to 800 nm.

19. The method of claim 1, which further comprises the step of rehydrating the formulation.

20. The method of claim 19, wherein the formulation is in the form of an aqueous suspension and further comprises an aqueous vehicle.

21. The method of claim 20, wherein the aqueous suspension further comprises an acoustically active gas.

22. The method of claim 1, wherein the stabilizing agent is a polymer, a lipid, a polymer-lipid conjugate, or a combination thereof.

23. The method of claim 22, wherein the stabilizing agent is a polymer.

24. The method of claim 23, wherein the polymer is selected from linear, branched and star structures.

25. The method of claim 24, wherein the polymer is a block copolymer.

26. The method of claim 25, wherein the polymer is a branched block copolymer selected from polyethylene glycol-polypropylene oxide, polyethylene glycol-polylactide, polyethylene glycol-polylactide-coglycolide, and polyethylene glycol-b-polycaprolactone copolymers.

27. The method of claim 25, wherein the polymer is a branched block copolymer having a central core and about 3 to 12 arms radiating therefrom.

28. The method of claim 27, wherein each arm comprises a block copolymer with an inner, more hydrophobic block and an outer, more hydrophilic block.

29. The method of claim 27, wherein each arm comprises a block copolymer with an inner, more hydrophilic block and an outer, more hydrophobic block.

30. The method of claim 27, wherein the branched block copolymer about 4 to about 8 arms.

31. The method of claim 24, wherein the polymer comprises repeating alkylene groups, wherein each alkylene group optionally contains from one to three heteroatoms selected from —O—, —N(R)— or —S(O)ₙ—, where R is hydrogen or alkyl and n is 0 to about 1000.

32. The method of claim 24, wherein the polymer is selected from polyalkylene oxides, polyalkyleneimines, polyalkylene amines, polyalkene sulfides, polyalkylene sulfonates, polyalkylene sulfones, poly(alkylenesulfonylalkyleneimine)s, polycaprolactones, polylactides, polyglycolides, and derivatives, mixtures and copolymers thereof.

33. The method of claim 24, wherein the polymer is selected from poloxamer, poloxamine, polyethylene glycol, polypropylene glycol, branched polyethylene imine, polyvinyl pyrrolidone, polylactide, poly(lactide-co-glycolide), polysorbate, polyethylene oxide, poly(ethylene oxide-co-propylene oxide), poly(oxyethylated) glycerol, poly(oxyethylated) sorbitol, poly(oxyethylated glucose), polymethyloxazoline, polyethyloxazoline, polyhydroxyethyloxazoline, polyhydroxypropyloxazoline, polyvinyl alcohol, poly(hydroxyalkylcarboxylic acid), polyhydroxyethyl acrylic acid, polyhydroxypropyl methacrylic acid, polyhydroxyvalerate, polyhydroxybutyrate, polyoxazolidine, polyaspartamide, polysialic acid, linear polypropylene imine, polyethylene sulfide, polypropylene sulfide, polyethylenesulfonate, polypropylenesulfonate, polyethylene sulfone, polyethylenesulfonylethyleneimine, polycaprolactone, polypropylene oxide, polyvinylmethylether, polyhydroxyethyl acrylate, polyhydroxypropyl methacrylate, polyphosphazene, and derivatives, mixtures and copolymers thereof.

34. The method of claim 33, wherein the polymer is selected from the group consisting of a polyethylene glycol and polypropylene glycol and copolymers thereof.

35. The method of claim 34, wherein the polymer is selected from branched polyethylene glycol, star polyethylene glycol, linear polyethylene glycol, and combinations thereof, and is optionally covalently bound to at least one phospholipid moiety.

36. The method of claim 34, wherein the polyethylene glycol is functionalized to contain at least one sulfhydryl, amino, lower alkoxy, carboxylate, or phosphonate moiety.

37. The method of claim 34, wherein the polyethylene glycol or polypropylene glycol contains a hydrolyzable linkage.

38. The method of claim 34, wherein the polyethylene glycol is bonded to a phospholipid moiety.

39. The method of claim 38, wherein the polyethylene glycol ranges in size from about 350 to 7000 daltons.

40. The method of claim 39, wherein the polyethylene glycol ranges in size from about 750 to 5000 daltons.

41. The method of claim 23, wherein the polymer is a polysorbate.

42. The method of claim 23, wherein the polymer is a polypeptide.

43. The method of claim 22, wherein the stabilizing agent is a lipid with a lipid to drug weight ratio less than 5:1, more preferably 3:1, most preferably less than 1:1.

44. The method of claim 43, wherein the lipid is selected from natural phospholipids, chemically and enzymatically modified phospholipids, and synthetic phospholipids.

45. The method of claim 44, wherein the lipid is a diacyl phospholipid.

46. The method of claim 45, wherein the lipid is selected from diacyl phosphatidylcholines, diacyl phosphatidylethanolamines, diacyl phosphatidylserines, diacyl phosphatidylinositols, diacyl phosphatidic acids, phosphorylated diacylglycerides, and combinations thereof.

47. The method of claim 44, wherein the lipid is a phosphorylated diacylglyceride.

48. The method of claim 47, wherein the phosphorylated diacylglyceride is selected from diolcoyl phosphatidylglycerol, palmitoyloleyl phosphatidylglycerol and combinations thereof.

49. The method of claim 44, wherein the lipid is a diacyl phosphatidylcholine.

50. The method of claim 49, wherein the diacyl phosphatidylcholine is selected from palmitoyloleoyl phosphatidylcholine, dioleoyl phosphatidylcholine, dilauroyl phosphatidylcholine, dimyristoyl phosphatidylcholine, dipalmitoyl phosphatidylcholine, distearoyl phosphatidylcholine, and mixtures thereof.

51. The method of claim 44, wherein the lipid is a diacyl phosphatidylethanolamine.

52. The method of claim 51, wherein the diacyl phosphatidylethanolamine is selected from dipalmitoyl phosphatidylethanolamine, 1-palmitoyl-2-oleoylphosphatidylethanolamine, dioleylphosphatidylethanolamine, and combinations thereof.

53. The method of claim 22, wherein the stabilizing agent is a polymer-lipid conjugate.

54. The method of claim 53 wherein the polymer is polyethylene glycol and the lipid is selected from phospholipids and fatty acids.

55 The method of claim 1, wherein the admixing step further comprises including an excipient.

56. The method of claim 55, wherein the excipient is selected from polyhydroxyalcohols, saccharides, liquid polyethylene glycols, propylene glycol, glycerol, ethyl alcohol, and combinations thereof.

57. The method of claim 1, wherein the admixing step further comprises including a targeting ligand.

58. The method of claim 57, wherein the targeting ligand targets cells or receptors associated with diseased tissue.

59. The method of claim 58, wherein the targeting ligand is selected from proteins, peptides, cytokines, growth factors, vitamins, vitamin analogues, polysaccharides, glycopeptides, glycoproteins, steroids, steroid analogs, hormones, cofactors, bioactive agents, genetic material, drug molecules, and antagonists of the GPIIBIIIA receptor of platelets.

60. The method of claim 1, wherein the therapeutic agent is an anti-cancer agent.

61. The method of claim 60, wherein the anti-cancer agent is selected from paclitaxel, docetaxel, camptothecin, and derivatives and analogs thereof.

62. The method of claim 1, wherein the therapeutic agent has limited water solubility.

63. The method of claim 62, wherein the ratio of the solubility of the therapeutic agent in the stabilizing agent to the solubility of the therapeutic agent in water is greater than about 1:1.

64. The method of claim 63, wherein the ratio is at least about 10:1.

65. A nanoparticulate formulation prepared according to the method of claim 1.

66. A method of producing a sterile pharmaceutical formulation comprising:
(a) admixing, in a first solvent and a second solvent, a drug, and a stabilizing agent that stabilizes the drug but does not covalently bind thereto; wherein the first solvent is an organic solvent having a freezing temperature with the range of about 0-25° C.;
(b) filter sterilizing the mixture; and
(c) removing the first solvent and second solvent in a manner effective to provide a dry formulation of the drug.

67. The method of claim 66, wherein the first solvent is selected from tert-butyl alcohol, cyclohexane, dimethyl carbonate, dimethyl sulfoxide, and acetic acid.

68. The method of claim 66, wherein the second solvent is selected from alkylated alcohols, ethers, acetone, alkanes, dimethyl sulfoxide, chloroform, cyclic hydrocarbons, toluene, benzene, N,N-dimethylformamide, and mixtures thereof.

69. The method of claim 66, wherein the admixing step further comprises including a water-soluble bulking agent.

70. The method of claim 69, wherein the water-soluble bulking agent is selected from sorbitol, mannitol, xylitol, hydrogenated starch hydrolysates, maltitol, lactitol, maltitol, hydrogenated isomaltulose, erythritol, inositol, sucrose, and trehalose.

71. The method of claim 66, wherein the admixing step further comprises including an excipient.

72. The method of claim 66, wherein the admixing step further comprises including a targeting ligand.

73. The method of claim 66, which further comprises the step of sonicating the formulation.

74. The method of claim 66, which further comprises the step of purging with an inert gas.

75. The method of claim 66, wherein the solvent is removed by lyophilization.

76. The method of claim 66, wherein the solvent is removed by spray drying.

77. The method of claim 66, wherein solvent removal step comprises rotary evaporation, thereby providing an agglomerated intermediate product, and wherein the method further comprises deagglomerating the intermediate product using a procedure effective to provide the dry formulation of the drug.

78. The method of claim 66, wherein the admixing step further comprises including a supercritical fluid.

79. The method of claim 78 wherein the solvent is removed by supercritical fluid processing.

80. A nanoparticulate formulation prepared according to the method of claim 66.

81. A method of producing a sterile pharmaceutical formulation comprising:
(a) admixing, in a first solvent and a second solvent, a drug, a stabilizing agent that stabilizes the drug but does not covalently bind thereto, and a water-soluble bulking agent; wherein the first solvent is an organic solvent having a freezing temperature with the range of about 0-25° C.;
(b) filter sterilizing the mixture; and
(c) removing the first solvent and second solvent in a manner effective to provide a dry formulation of the drug.

82. The method of claim 81, wherein the first solvent is selected from tert-butyl alcohol, cyclohexane, dimethyl carbonate, dimethyl sulfoxide, and acetic acid.

83. The method of claim 81, wherein the second solvent is selected from alkylated alcohols, ethers, acetone, alkanes, dimethyl sulfoxide, chloroform, cyclic hydrocarbons, toluene, benzene, N,N-dimethylformamide, and mixtures thereof.

84. The method of claim 81, wherein the water-soluble bulking agent is selected from sorbitol, mannitol, xylitol, hydrogenated starch hydrolysates, maltitol, lactitol, maltitol, hydrogenated isomaltulose, erythritol, inositol, sucrose, and trehalose.

85. The method of claim 81, wherein the admixing step further comprises including an excipient.

86. The method of claim 81, wherein the admixing step further comprises including a targeting ligand.

87. The method of claim 81, which further comprises the step of sonicating the formulation.

88. The method of claim 81, which further comprises the step of purging with an inert gas.

89. The method of claim 81, wherein the solvent is removed by lyophilization.

90. The method of claim 81, wherein the solvent is removed by spray drying.

91. The method of claim 81, wherein solvent removal step comprises rotary evaporation, thereby providing an agglomerated intermediate product, and wherein the method further comprises deagglomerating the intermediate product using a procedure effective to provide the dry formulation of the drug.

92. The method of claim 81, wherein the admixing step further comprises including a supercritical fluid.

93. The method of claim 92 wherein the solvent is removed by supercritical fluid processing.

94. A nanoparticulate formulation prepared according to the method of claim 81.

95. An anisotropic nanoparticle or microparticle formulation of a drug comprising one or more stabilizing agents wherein said particles have a rod-like appearance and the particles are at least two times longer than they are wide.

96. The nanoparticle of claim 95, wherein the stabilizing agent comprises a polymer.

97. The nanoparticle of claim 96 wherein the stabilizing agent comprises a branching block polymer.
